SEQUENCE LISTING (56 sequences)

SEQ ID NO: 1 - protein, 30 aa, synthetic construct
  VARIANT 1: note = X0 = any proteinogenic amino acid sequence and can range from 1-10 amino acids
  VARIANT 2: note = any amino acid
  VARIANT 5: note = any amino acid
  VARIANT 4: note = X4 = K, R, Q, N, D, E, A, L, and M
  VARIANT 8: note = X7 = K, R, Q, N, D, E, A, L, and M
  VARIANT 11: note = X10 = K, R, Q, N, D, E, A, L, and M
  VARIANT 12: note = X11 = independently any proteinogenic amino acid, wherein 1, 2, 3, 4, or 5 proteinogenic amino acids may be inserted additionally
  VARIANT 14: note = X13 = independently any proteinogenic amino acid, wherein 1, 2, 3, 4, or 5 proteinogenic amino acids may be inserted additionally
  VARIANT 15: note = X14 = K, R, Q, N, D, E, A, L, or M
  VARIANT 16: note = X15 = K, R, Q, N, D, E, A, L, or M
  VARIANT 17: note = X16 = I, E, and T
  VARIANT 20: note = X19 = K, R, Q, N, D, E, A, L, or M
  VARIANT 23: note = X22 = K, R, Q, N, D, E, A, L, or M
  VARIANT 24: note = X23 = A, K, T, R, Q, N, D, E, A, L, or M
  VARIANT 27: note = X26 = K, R, Q, N, D, E, A, L, or M
  VARIANT 30: note = any proteinogenic amino acid sequence and can range from 1-20 amino acids
  VARIANT 13: note = X12 = independently any proteinogenic amino acid, wherein 1, 2, 3, 4, or 5 proteinogenic amino acids may be inserted additionally
XXLXXLVXLLXXXXXXXLLXALXXLAXIAX

SEQ ID NO: 2 - protein, 41 aa, synthetic construct
NEQIQAVIDAGALEKLEQLQSHENEKIQKEAQEALEKLQSH

SEQ ID NO: 3 - protein, 10 aa, synthetic construct
  VARIANT 1: note = X = K, R, Q, N, D, E, A, L, and M
XEQIQAVIDA

SEQ ID NO: 4 - protein, 10 aa, synthetic construct
ALPALVQLLS

SEQ ID NO: 5 - protein, 17 aa, synthetic construct
  VARIANT 3: note = X1= any amino acid
  VARIANT 6: note = X = any amino acid
  VARIANT 7: note = X = any amino acid
  VARIANT 10: note = X = any amino acid
  VARIANT 13: note = X = any amino acid
  VARIANT 17: note = X = any amino acid
NEXILXXALXALXNIAX

SEQ ID NO: 6 - protein, 32 aa, synthetic construct
PDLPKLVKLLKSSNEEILLKALRALAEIASGG

SEQ ID NO: 7 - protein, 32 aa, synthetic construct
PDLPKLVKLLKSSNEEILLKALKALAEIASGG

SEQ ID NO: 8 - protein, 32 aa, synthetic construct
GALPALVQLLSSPDEETLLKALKTLAEIASGG

SEQ ID NO: 9 - protein, 32 aa, synthetic construct
PDLPKLVKLLKSSDEETLLKALRTLAEIASGG

SEQ ID NO: 10 - protein, 32 aa, synthetic construct
PDLPKLVKLLKSSDEETLLKALKTLAEIASGG

SEQ ID NO: 11 - protein, 32 aa, synthetic construct
PDLPKLVKLLKSSDEKTLLEALKTLAEIASGG

SEQ ID NO: 12 - protein, 32 aa, synthetic construct
ELPQMTQQLNSDDMQEQLSATVKFRQILSREH

SEQ ID NO: 13 - protein, 43 aa, synthetic construct
MRGSHHHHHHGSSLNELVKQLNSDDQKQLKEAAQKLRQLASDG

SEQ ID NO: 14 - protein, 44 aa, synthetic construct
MRGSHHHHHHGSELPQMTQQLNSDDMQEQLSATVKFRQILSRDG

SEQ ID NO: 15 - protein, 43 aa, synthetic construct
MRGSHHHHHHGSELPQMTQQLNSDDMQEQLSATRKFSQILSDG

SEQ ID NO: 16 - protein, 43 aa, synthetic construct
MRGSHHHHHHGSELPQMVQQINSPDQQELQSALRKLSQIASGG

SEQ ID NO: 17 - protein, 13 aa, synthetic construct
PDLPKLVKLLKSS

SEQ ID NO: 18 - protein, 13 aa, synthetic construct
PDLPKLVKLLKSS

SEQ ID NO: 19 - protein, 13 aa, synthetic construct
PDLPKLVKLLKSS

SEQ ID NO: 20 - protein, 13 aa, synthetic construct
PDLPKLVKLLKSS

SEQ ID NO: 21 - protein, 13 aa, synthetic construct
PDLPKLVKLLKSS

SEQ ID NO: 22 - protein, 13 aa, synthetic construct
GALPALVQLLSSP

SEQ ID NO: 23 - protein, 13 aa, synthetic construct
GALPALVQLLSSP

SEQ ID NO: 24 - protein, 13 aa, synthetic construct
GELPQMVQQLNSP

SEQ ID NO: 25 - protein, 17 aa, synthetic construct
NEEILLKALRALAEIAS

SEQ ID NO: 26 - protein, 17 aa, synthetic construct
NEEILLKALKALAEIAS

SEQ ID NO: 27 - protein, 17 aa, synthetic construct
DEETLLKALRTLAEIAS

SEQ ID NO: 28 - protein, 17 aa, synthetic construct
DEKTLLEALKTLAEIAS

SEQ ID NO: 29 - protein, 17 aa, synthetic construct
DEETLLKALKTLAEIAS

SEQ ID NO: 30 - protein, 17 aa, synthetic construct
DEETLLKALKTLAEIAS

SEQ ID NO: 31 - protein, 17 aa, synthetic construct
NEQILQEALWALSNIAS

SEQ ID NO: 32 - protein, 17 aa, synthetic construct
DQQELQSALRKLSQIAS

SEQ ID NO: 33 - DNA, 30 bp, synthetic construct
  source 1..30: mol_type = other DNA
aaagctcttcacagggcgcccttccagccc

SEQ ID NO: 34 - DNA, 20 bp, synthetic construct
  source 1..20: mol_type = other DNA
gctttgttagcagccggatc

SEQ ID NO: 35 - DNA, 51 bp, synthetic construct
  source 1..51: mol_type = other DNA
cgaaagcagcggcctggaagtgctgtttcagggtccgagaagagccatggc

SEQ ID NO: 36 - DNA, 51 bp, synthetic construct
  source 1..51: mol_type = other DNA
gccatggctcttctcggaccctgaaacagcacttccaggccgctgctttcg

SEQ ID NO: 37 - DNA, 62 bp, synthetic construct
  source 1..62: mol_type = other DNA
aaagctcttcaccgggatccaaaagtggtctcggcgccggctcgaagggggaagaagataac

SEQ ID NO: 38 - DNA, 37 bp, synthetic construct
  source 1..37: mol_type = other DNA
aaaagatctttattacttataaagctcatccatgccc

SEQ ID NO: 39 - DNA, 32 bp, synthetic construct
  source 1..32: mol_type = other DNA
aaagctcttcaaccgcttgcaatctgtgagag

SEQ ID NO: 40 - DNA, 34 bp, synthetic construct
  source 1..34: mol_type = other DNA
aaagctcttcaggcggtaacgagcagattcaggc

SEQ ID NO: 41 - DNA, 54 bp, synthetic construct
  source 1..54: mol_type = other DNA
aaagctcttcagtgaagttactgaaaagctctaacgaacagattctccaagagg

SEQ ID NO: 42 - DNA, 55 bp, synthetic construct
  source 1..55: mol_type = other DNA
aaagctcttcacaccagtttaggcagatccggaccctggaagtacaggttttcgc

SEQ ID NO: 43 - DNA, 57 bp, synthetic construct
  source 1..57: mol_type = other DNA
aaagctcttcactgcgtgcactcgctgaaattgccagcggcggtaacgagcagattc

SEQ ID NO: 44 - DNA, 60 bp, synthetic construct
  source 1..60: mol_type = other DNA
aaagctcttcacagcgctttcagcaggatttcctcgttagagcttttcagtaacttcacc

SEQ ID NO: 45 - DNA, 57 bp, synthetic construct
  source 1..57: mol_type = other DNA
aaagctcttcactgaaggcactcgctgaaattgccagcggcggtaacgagcagattc

SEQ ID NO: 46 - DNA, 57 bp, synthetic construct
  source 1..57: mol_type = other DNA
aaagctcttcactgaagacactcgctgaaattgccagcggcggtaacgagcagattc

SEQ ID NO: 47 - DNA, 59 bp, synthetic construct
  source 1..59: mol_type = other DNA
aaagctcttcacagcgctttcagcagggtttcctcatcaggtgacgaaagcaattggac

SEQ ID NO: 48 - DNA, 57 bp, synthetic construct
  source 1..57: mol_type = other DNA
aaagctcttcactgcgtacactcgctgaaattgccagcggcggtaacgagcagattc

SEQ ID NO: 49 - DNA, 60 bp, synthetic construct
  source 1..60: mol_type = other DNA
aaagctcttcacagcgctttcagcagggtttcctcatcagagcttttcagtaacttcacc

SEQ ID NO: 50 - DNA, 60 bp, synthetic construct
  source 1..60: mol_type = other DNA
aaagctcttcacagcgcttccagcagggttttctcatcagagcttttcagtaacttcacc

SEQ ID NO: 51 - DNA, 31 bp, synthetic construct
  source 1..31: mol_type = other DNA
aaagctcttcacccaccagaggcaatgttag

SEQ ID NO: 52 - DNA, 35 bp, synthetic construct
  source 1..35: mol_type = other DNA
aaagctcttcagggaatgagcaaatccaagccgtg

SEQ ID NO: 53 - DNA, 20 bp, synthetic construct
  source 1..20: mol_type = other DNA
taatacgactcactataggg

SEQ ID NO: 54 - DNA, 19 bp, synthetic construct
  source 1..19: mol_type = other DNA
gctagttattgctcagcgg

SEQ ID NO: 55 - protein, 7 aa, synthetic construct
  VARIANT 6: note = X=not Cysteine
ALLAAXC

SEQ ID NO: 56 - protein, 30 aa, synthetic construct
  VARIANT 1: note = any proteinogenic amino acid sequence and can range from 1-10 amino acids
  VARIANT 2: note = X1 = any proteinogenic amino acid
  VARIANT 4: note = X3 = any amino acid
  VARIANT 5: note = X4 = K, A, E
  VARIANT 8: note = X7= K, E
  VARIANT 11: note = X11 = K, E, S
  VARIANT 13: note = X12= any amino acid
  VARIANT 14: note = X13= N, D
  VARIANT 16: note = X15 = E, K
  VARIANT 17: note = X16 = I, T
  VARIANT 20: note = X19 = K, E
  VARIANT 23: note = X22 = K, R
  VARIANT 24: note = X23 = A, T
  VARIANT 27: note = X26= E, Q
  VARIANT 30: note = any proteinogenic amino acid sequence and can range from 1-20 amino acids
XXLXXLVXLLXSXXEXXLLXALXXLAXIAX

(19) United States
(12) Patent Application Publication
(10) Pub. No.: US 2025/0368704 A1
(43) Pub. Date: Dec. 4, 2025

(54) STABILIZING N-CAP SEQUENCES FOR ARMADILLO REPEAT PROTEINS

(71) Applicant: UNIVERSITÄT ZÜRICH, Zürich (CH)

(72) Inventors: Andreas PLÜCKTHUN, Zürich (CH); Erich MICHEL, Zürich (CH)

(73) Assignee: UNIVERSITÄT ZÜRICH, Zürich (CH)

(21) Appl. No.: 18/721,904

(22) PCT Filed: Jan. 9, 2023
(86) PCT No.: PCT/EP2023/050328

§ 371 Date: Jun. 20, 2024

(30) Foreign Application Priority Data
Jan. 7, 2022 (EP) 22150592.8

(51) Int. Cl. C07K 14/47

(52) U.S. Cl. CPC C07K 14/4703

(57) ABSTRACT

The present invention relates to N-terminal cap sequences which stabilize armadillo repeat proteins.

### CROSS-REFERENCE TO RELATED APPLICATIONS

This is the U.S. National Stage of International Patent Application No. PCT/EP2023/050328 filed on Jan. 9, 2023, which claims the benefit of European Patent Application EP22150592.8 filed on Jan. 7, 2022, which is incorporated by reference herein.

### INCORPORATION BY REFERENCE OF SEQUENCE LISTING

The nucleic and/or amino acid sequences provided herewith are shown using standard letter abbreviations for nucleotide bases, and one letter code for amino acids, as defined in with 37 CFR 1.831 through 37 CFR 1.835. Only one strand of each nucleic acid sequence is shown, but the complementary strand is understood as included by any reference to the displayed strand.

The Sequence Listing is submitted as an XML file named 95083_303_77_SEQ_LISTING created Feb. 2, 2025, about 66000 Bytes, which is incorporated by reference herein in its entirety.

### FIELD

The present invention relates to N-terminal cap sequences which stabilize armadillo repeat proteins.

### BACKGROUND OF THE INVENTION

The need for binding proteins that recognize linear or structural epitopes with high affinity and specificity is ever-increasing. These binding proteins are used as therapeutics, diagnostics and research reagents. Nowadays, most commercially available protein binders, in all three categories, are based on the antibody scaffold; however, alternative scaffolds with attractive properties are emerging. A particularly interesting scaffold for the recognition of linear epitopes is provided by Armadillo repeat proteins (ArmRPs), an abundant eukaryotic protein family involved in a wide variety of biological functions that include transcription regulation, nuclear transport, and cellular adhesion, amongst others.

Naturally occurring ArmRPs (nArmRPs) are typically composed of around 8-12 internal repeats, which are flanked by N- and C-terminal capping repeats. Each internal module contains around 42 amino acids that constitute three helices H1, H2, and H3, which fold into a right-handed triangular staircase. The assembly of multiple repeats thus generates an elongated, right-handed superhelical protein molecule that exposes a concave binding surface composed of adjacent helices H3. This surface interacts with polypeptide segments in an extended conformation. This recognition involves specific interactions between the bound peptide sidechains and the binding surface of the nArmRPs and is further enhanced by hydrogen bonds between the peptide backbone and conserved asparagine residues in helices H3. In a first approximation 2-3 amino acids of the peptide are recognized per internal module; however, this modular peptide-binding mode is less regular in nArmRPs and typically shows an alteration between short bound and unbound peptide stretches. Therefore, in nArmRPs, deviations from an ideal binding stoichiometry of two target amino acids per module are frequently observed.

The objective of the present invention is to provide means and methods to provide N-terminal cap sequences which stabilize armadillo repeat proteins. This objective is attained by the subject-matter of the independent claims of the present specification, with further advantageous embodiments described in the dependent claims, examples, figures and general description of this specification.

### SUMMARY OF THE INVENTION

Designed ArmRPs (dArmRPs) have been engineered with the aim to create sequence-specific peptide-binding scaffolds that feature consecutive peptide recognition and an ideal stoichiometry of exactly two amino acids of the target peptide recognized per internal module. So-called C-type internal modules of the dArmRPs were obtained from a consensus design approach based on more than 240 input sequences from the importin-α and β-catenin/plakoglobin superfamilies. Further computational optimization of three hydrophobic core positions for improved packing in the C-type consensus design and mutation of two lysine residues to glutamines to prevent electrostatic repulsions provided the M-type internal module.

The significant contribution of capping repeats to the overall protein stability and to prevent aggregation has been shown previously for designed Ankyrin repeat proteins (DARPins). Thus, particular attention in the capping repeat design is crucial for engineering of repeat proteins with desirable properties such as high stability and solubility and no or little tendency to aggregate. The C-terminal C_{AI}-capping repeat for dArmRPs was designed by replacing hydrophobic surface-exposed residues of the C-type internal module with hydrophilic ones, using guidance from available structural and sequence alignment data. The C_{AII}-cap was subsequently generated by introducing two mutations near the C-terminus, which improved packing and solubility. Moreover, replacing the C_{AI}-cap with the C_{AII}-cap in dArmRPs with four internal M modules significantly increased the melting temperature by ca. 7° C. and the transition midpoint in GdnHCl-induced unfolding by more than ca. 0.5 M GdnHCl.

Previous data on the N-terminal domain boundaries of N-capping repeats in dArmRPs from limited proteolysis experiments and sequence alignments did not provide a clear boundary definition of the stable portion of the N-capping repeat. Moreover, nArmRP crystal structures only provided resolved structural information for helices H2 and H3 in the N-cap, probably due to conformational dynamics. Therefore, invisible residues were not considered as parts of the folded N-capping domain, and the N-capping domain was defined to comprise only helices H2 and H3.

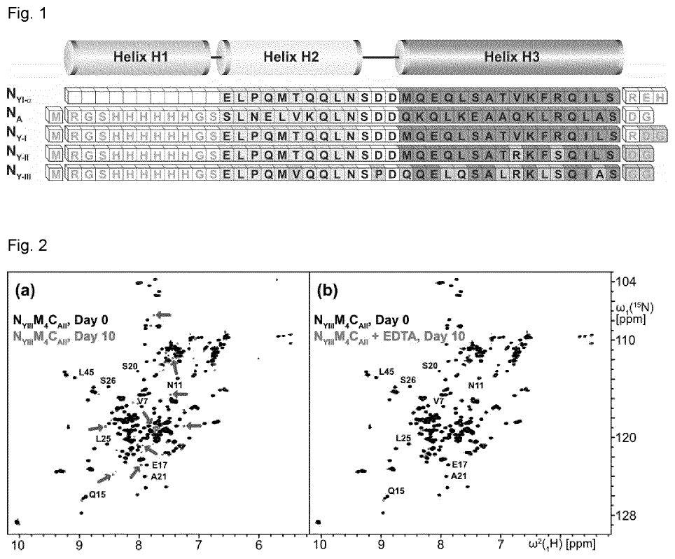

The first design of an N-capping repeat (N_{A}), which was based on optimization of surface-exposed residues in the C-type internal module (FIG. 1), resulted in very low dArmRP solubility and expression yields. An alternative N-cap design (N_{YI}) used residues E88-H119 of yeast importin-α as a starting scaffold and further introduced the R117D and E118G mutations in the linker between helix H3 of the N-cap and helix H1 of the next internal module. This N_{YI}-cap provided enhanced solubility and expression yields; however, MD simulations and NMR experiments suggested significant flexibility in the N_{YI}-cap, which was addressed in the N_{YII}-cap by mutations V24R and R27S and deletion of R32 (FIG. 1) to match the linker length between internal M-modules. Exchanging the N_{YI}-cap with the N_{YII}-cap in dArmRPs with four internal M modules showed rather modest increases of ca. 2° C. in the melting temperature and 0.1-0.15 M GdnHCl in the transition midpoint in GdnHCl-induced unfolding.

Despite the improved features, crystal structures of dArmRPs containing the N_{YII}-cap revealed domain swapping of the N_{YII}-cap due to formation of a continuous α-helix comprising H3 of the N_{YII}-cap and H1 of the first M module. To further stabilize the N_{YII}-cap and to avoid domain-swapping, the obtained crystal structures served as templates for a structure-based re-engineering of the N_{YII}-cap: the D41G mutation aimed at minimizing the helix propensity of the residues between N-cap and internal M module and thus to suppress formation of a continuous helix comprised of helices H3 and H1; mutations T17V, Q28L, T32L, F35L, L39A intended to improve packing of the hydrophobic core, M25Q and L29Q lowered the hydrophobicity of surface-exposed residues, and D23P enhanced the helix-breaking properties between helices H1 and H2 (FIG. 1). Overall, replacing the N_{YII}-cap with the N_{YIII}-cap increased the melting temperature by 4.5° C. and the transition midpoint in GdnHCl-induced unfolding by 0.2 M GdnHCl.

The successive engineering of the N-cap from the first N_{YI}-cap to the most recent N_{YII}-cap provided a combined stabilization that resulted in increases by ca. 6.5° C. in thermal unfolding and 0.3-0.35 M GdnHCl in denaturant-induced unfolding experiments. Despite these stability improvements, the inventors now provide evidence that the N_{YIII}-cap is still considerably unstable and shows significant local unfolding, which facilitates proteolytic degradation and aggregation. To overcome these undesirable features and to provide a more robust N-cap, the inventors report the engineering of significantly stabilized N-cap versions by combining consensus design and computational optimization and provide experimental evidence that highlights the obtained stability improvement.

A first aspect of the invention relates to an armadillo repeat protein comprising or essentially consisting of
a. an N-terminal cap sequence;
b. a C-terminal cap sequence; and
c. a plurality of armadillo repeats,
   wherein each armadillo repeat comprises from N-terminus to C-terminus three helices a, b, and c, wherein the helices a and b are connected via a loop a/b, and the helices b and c are connected via a loop b/c, and wherein two armadillo repeats are connected via a loop c/a;
characterized in that
   the N-terminal cap sequence consists of the sequence X₀X₁LX₃X₄LVX₇LLX₁₀X₁₁X₁₂X₁₃X₁₄X₁₅X₁₆LX₁₉ALX₂₂X₂₃LAX₂₆IAX₂₉ (SEQ ID NO: 1).

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term armadillo repeat protein in the context of the present specification relates to a protein of UniProt-ID Q02821 (importin subunit alpha from Baker's yeast) or a derivative thereof. The term armadillo repeat protein refers to a polypeptide comprising at least one armadillo repeat, wherein an armadillo repeat is characterized by three alpha helices in a triangular arrangement.

### Sequences

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

Reference to identical sequences without specification of a percentage value implies 100% identical sequences (i.e. the same sequence).

### General Biochemistry: Peptides, Amino Acid Sequences

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof. The term “polypeptides” and “protein” are used interchangeably herein and include proteins and fragments thereof. Polypeptides are disclosed herein as amino acid residue sequences.

The term peptide in the context of the present specification relates to a molecule consisting of up to 50 amino acids, in particular 8 to 30 amino acids, more particularly 8 to 15 amino acids, that form a linear chain wherein the amino acids are connected by peptide bonds.

Amino acid residue sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids. Sequences are written left to right in the direction from the amino to the carboxy terminus. In accordance with standard nomenclature, amino acid residue sequences are denominated by either a three letter or a single letter code as indicated as follows. The 20 proteinogenic amino acids are: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

### DETAILED DESCRIPTION OF THE INVENTION

A first aspect of the invention relates to an armadillo repeat protein comprising or essentially consisting of (from N- to C-terminus)
a. an N-terminal cap sequence;
b. a plurality of armadillo repeats,
   wherein each armadillo repeat comprises from N-terminus to C-terminus three helices a, b, and c, wherein the helices a and b are connected via a loop a/b, and the helices b and c are connected via a loop b/c, and wherein two armadillo repeats are connected via a loop c/a; and
c. a C-terminal cap sequence;
wherein
   the C-terminal cap sequence consists of a sequence NEQIQAVIDAGALEKLEQLQSHENEKIQKEAQEALEKLQSH (SEQ ID NO: 2);
   helix a consists of a sequence X⁷EQIQAVIDA (SEQ ID NO: 3);
   loop a/b consists of a single glycine G;
   helix b consists of a sequence ALPALVQLLS (SEQ ID NO: 4),
   loop b/c consists of a sequence serine proline SP;
   helix c consists of a sequence NEX¹ILX²X³ALX⁴ALX⁵NIAX⁶ (SEQ ID NO: 5); and
   loop c/a consist of 1 to 9 proteinogenic amino acids;
wherein each X¹-X⁷ can be any proteinogenic amino acid provided that the amino acid does not prevent helix formation of helix a and c;
wherein
   1, 2, or 3 amino acids per armadillo repeat (meaning in each armadillo repeat unit) may be inserted at the beginning or the end of helices (as a helix extension) or inside the loops, and/or
   1, 2, or 3 amino acids per armadillo repeat and per C-terminal cap sequence may be exchanged (meaning 1, 2, or 3 amino acid substitutions per armadillo repeat unit and/or per C-terminal cap sequence), particularly according to the substitution rules given below;
the armadillo repeat protein being characterized in that
   the N-terminal cap sequence consists of the sequence X₀X₁LX₃X₄LVX₇LLX₁₀X₁₁X₁₂X₁₃X₁₄X₁₅X₁₆LLX₁₉ALX₂X₂₃LAX₂₆IAX₂₉ (SEQ ID NO: 1);
   wherein
   X₀: any proteinogenic amino acid sequence of 1-10 amino acids, wherein the sequence is capable of forming a helix, also called N-terminal helix extension (which is any sequence that causes the first helix of the N-cap to extend in length at its N-terminal end)
   X₁: any proteinogenic amino acid, particularly an amino acid selected from D, E, and A;
   X₃: any proteinogenic amino acid, particularly P;
   X₄: an amino acid selected from K, R, Q, N, D, E, A, L, and M;
   X₇: an amino acid selected from K, R, Q, N, D, E, A, L, and M;
   X₁₀: an amino acid selected from K, R, Q, N, D, E, A, L, and M;
   X₁₁₋₁₃: independently any proteinogenic amino acid, wherein 1, 2, 3, 4, or 5 proteinogenic amino acids may be inserted additionally into X₁₁₋₁₃, particularly X₁₁₋₁₃ are independently selected from S, T, G, P, N, and D;
   X₁₄: an amino acid selected from K, R, Q, N, D, E, A, L, and M;
   X₁₅: an amino acid selected from K, R, Q, N, D, E, A, L, and M;
   X₁₆: an amino acid selected from I, E, and T;
   X₁₉: an amino acid selected from K, R, Q, N, D, E, A, L, and M;
   X₂₂: an amino acid selected from K, R, Q, N, D, E, A, L, and M;
   X₂₃: an amino acid selected from A, K, T, R, Q, N, D, E, A, L, and M;
   X₂₆: an amino acid selected from K, R, Q, N, D, E, A, L, and M;
   X₂₉: 1-20, particularly 1-10, amino acids independently selected from any proteinogenic amino acid provided that the amino acid does not prevent loop formation.

### Substitution Rules

a. glycine (G), serine(S), and alanine (A) are interchangeable; valine (V), leucine (L), and isoleucine (I) are interchangeable, A and V are interchangeable;
b. tryptophan (W) and phenylalanine (F) are interchangeable, tyrosine (Y) and F are interchangeable;
c. serine(S) and threonine (T) are interchangeable;
d. aspartic acid (D) and glutamic acid (E) are interchangeable
e. asparagine (N) and glutamine (Q) are interchangeable; N and S are interchangeable; N and D are interchangeable; E and Q are interchangeable;
f. methionine (M) and Q are interchangeable;
g. cysteine (C), A, V and S are interchangeable;
h. proline (P), G, S and A are interchangeable;
i. arginine (R) and lysine (K) are interchangeable;
j. salt bridge partners are interchangeable, meaning that K, R or H is exchanged for D or E, when also D or E is exchanged for K, R or H at the opposite position of the salt bridge.

A residue X which does not prevent helix formation is an amino acid which at the position it is inserted integrates into the secondary helix structure without disturbing the helical structure. In certain embodiments, the “proteinogenic amino acid that does not prevent helix formation of helix a and c” is any proteinogenic amino acid except proline (P), meaning that the amino acid is selected from A, G, V, L, I, H, K, R, S, T, N, Q, D, E, F, W, Y, C, M.

A residue X which does not prevent helix formation is an amino acid which, at the position into which it is inserted, integrates into the loop without disturbing the loop structure. In certain embodiments, the “proteinogenic amino acid that does not prevent loop formation” can be any proteinogenic amino acid.

In certain embodiments, the armadillo repeat protein additionally comprises an N-terminal tag sequence.

In certain embodiments, the N-terminal cap consists of the sequence X₀X₁LX₃X₄LVX₇LLX₁₀X₁₁X₁₂X₁₃X₁₄X₁₅X₁₆LLX₁₉ALX₂₂X₂₃LAX₂₆IAX₂₉ (SEQ ID NO: 1), wherein
X₀: any proteinogenic amino acid sequence of 1-10 amino acids, wherein the sequence is capable of forming a helix;
X₁: any proteinogenic amino acid, particularly an amino acid selected from D and A;
X₃: any proteinogenic amino acid, particularly P;
X₄: an amino acid selected from K, Q, A, and E;
X₇: an amino acid selected from K and E;
X₁₀: an amino acid selected from K, S, N, A, and E;
X₁₁₋₁₃: independently any proteinogenic amino acid, wherein 1, 2, 3, 4, or 5 proteinogenic amino acids may be inserted additionally in X₁₁₋₁₃, particularly
   X₁₁ is selected from S, G, D and N,
   X₁₂ is selected from S, T, G, P, N and D, and
   X₁₃ is selected from N and D;
X₁₄: an amino acid selected from K, R, Q, E, A, and L;
X₁₅: an amino acid selected from K, R, Q, E, A, and L;
X₁₆: an amino acid selected from I, E, and T;
X₁₉: an amino acid selected from K, R, Q, E, A, and L;
X₂₂: an amino acid selected from K, R, Q, E, A, and L;
X₂₃: an amino acid selected from K, R, Q, E, A, L and T;
X₂₆: an amino acid selected from K, R, Q, E, A, and L;
X₂₉: 1-20, particularly 1-10, amino acids independently selected from any proteinogenic amino acid provided that the amino acid does not prevent loop formation.

In certain embodiments, the N-terminal cap consists of the sequence X₀X₁LX₃X₄LVX₇LLX₁₀SX₁₂X₁₃EX₁₅X₁₆LLX₁₉ALX₂₂X₂₃LAX₂₆IAX₂₉ (SEQ ID NO: 56), wherein
X₀: any proteinogenic amino acid sequence of 1-10 amino acids, wherein the sequence is capable of forming a helix;
X₁: any proteinogenic amino acid selected from D and A;
X₃: any proteinogenic amino acid, particularly P;
X₄: an amino acid selected from K, A, and E;
X₇: an amino acid selected from K and E;
X₁₀: an amino acid selected from K, E, and S;
X₁₂: any proteinogenic amino acid provided that the amino acid does not prevent loop formation, particularly S;
X₁₃: an amino acid selected from N and D;
X₁₅: an amino acid selected from E and K;
X₁₆: an amino acid selected from I and T;
X₁₉: an amino acid selected from K and E;
X₂₂: an amino acid selected from K and R;
X₂₃: an amino acid selected from A and T;
X₂₆: an amino acid selected from E and Q;
X₂₉: 1-20, particularly 1-10, amino acids independently selected from any proteinogenic amino acid provided that the amino acid does not prevent loop formation.

In certain embodiments, the N-terminal cap sequence is selected from a sequence in the following table

| N-Cap | | |
|---|---|---|
| Variant | Sequence | SEQ ID NO: |
| NA4 | PDLPKLVKLLKSSNEEILLKALRALAEIASGG | 6 |
| NA5 | PDLPKLVKLLKSSNEEILLKALKALAEIASGG | 7 |
| NA6 | GALPALVQLLSSPDEETLLKALKTLAEIASGG | 8 |
| NA7 | PDLPKLVKLLKSSDEETLLKALRTLAEIASGG | 9 |
| NA8 | PDLPKLVKLLKSSDEETLLKALKTLAEIASGG | 10 |
| NA9 | PDLPKLVKLLKSSDEKTLLEALKTLAEIASGG | 11 |

wherein optionally, the N-terminal cap sequence may be varied:
   a total of 1, 2, or 3 amino acids per N-terminal cap sequence may be inserted, and/or
   a total of 1, 2, or 3 amino acids per N-terminal cap sequence may be removed, and/or
   1, 2, or 3 amino acids per N-terminal cap sequence may be exchanged. In certain embodiments, the exchange is according to the substitution rules listed above.

In certain embodiments, the N-terminal cap sequence is selected from a sequence in the table above without any variation.

Wherever alternatives for single separable features such as, for example, a helix or loop sequence or a definition of a residue are laid out herein as “embodiments”, it is to be understood that such alternatives may be combined freely to form discrete embodiments of the invention disclosed herein. Thus, any of the alternative embodiments for a helix or loop sequence may be combined with any of the alternative embodiments of a definition of a residue mentioned herein.

### DETAILED DESCRIPTION OF FIGURES

FIG. 1 shows previous generations of N-caps for dArmRPs. Sequences of previously engineered N-cap variants are shown. Residues in yellow and green boxes indicate helices H2 and H3, respectively. Helix H1 is shown for its position in internal Arm repeats, there is no indication that the His tag would form a helix. Light blue boxes indicate modified positions. N_{YI-α}: yeast importin-α; N_{A}: artificial cap derived from consensus design and previous computational optimization; N_{Y-I}, N_{Y-II} and N_{YIII}: first, second, and third generation caps derived from yeast importin-α and computational optimization. The sequences depicted in this figure relate to the SEQ ID NOs: 12-16.

FIG. 2 shows NMR analysis of NYIIIM4CAII revealing sample instability. Superpositions of 2D [¹⁵N,¹H]-HSQC spectra of 100 μM N_{YIII}M₄C_{AII} in PBS buffer at pH 7 after 0 and 10 days of incubation at 37° C. measured either in the absence (a) or presence (b) of 250 μM EDTA. Black and red resonances indicate spectra after 0 and 10 days, respectively, while blue arrows exemplify additional signals that appear after 10 days. The assignments of some signals are indicated for orientation. All spectra were recorded at 37° C. and 600 MHz.

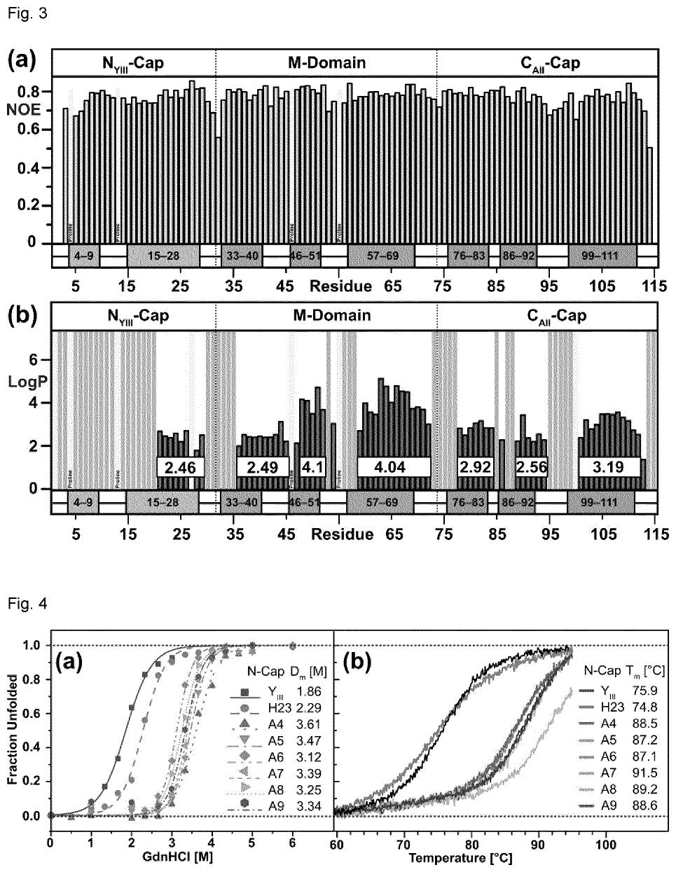

FIG. 3 shows conformational amide bond mobility and hydrogen exchange analysis for N_{YIII}MC_{AII} at pH 5.5. (a) Heteronuclear 2D ¹⁵N{¹H}-NOE values determined for individual backbone amide bonds in N_{YIII}MC_{AII} are plotted against the sequence. Colored boxes indicate helical segments in the N_{YIII}-cap (blue), M module (orange) and C_{AII}-cap (green) as determined from the secondary shift analysis. (b) Logarithm of protection factors (log P) obtained from the hydrogen exchange analysis of individual residues in N_{YIII}MC_{AII} plotted against the sequence. Grey bars indicate residues that exchange too fast to provide measurable P values while yellow bars indicate Proline residues or residues with overlapping amide resonances for which no P values could be obtained. Numbers in white boxes on red bars indicate averaged log P values for particular structural elements. All measurements were recorded at 20° C. on a 600 MHz spectrometer using 100 μM N_{YIII}MC_{AII} in 20 mM sodium phosphate at pH 5.5, containing 50 mM sodium chloride.

FIG. 4 shows denaturant-induced and thermal unfolding analysis of NMC constructs with different N-caps. (a) Guanidine hydrochloride (GdnHCl)-induced unfolding and (b) thermal unfolding curves of the different NMC proteins containing either newly designed N-caps or the original N_{YIII}-cap. Protein unfolding was monitored by following the CD signal at 222 nm. The obtained denaturation midpoint concentrations of GdnHCl, Dₘ, and melting temperatures Tₘ are indicated for each N-cap variant.

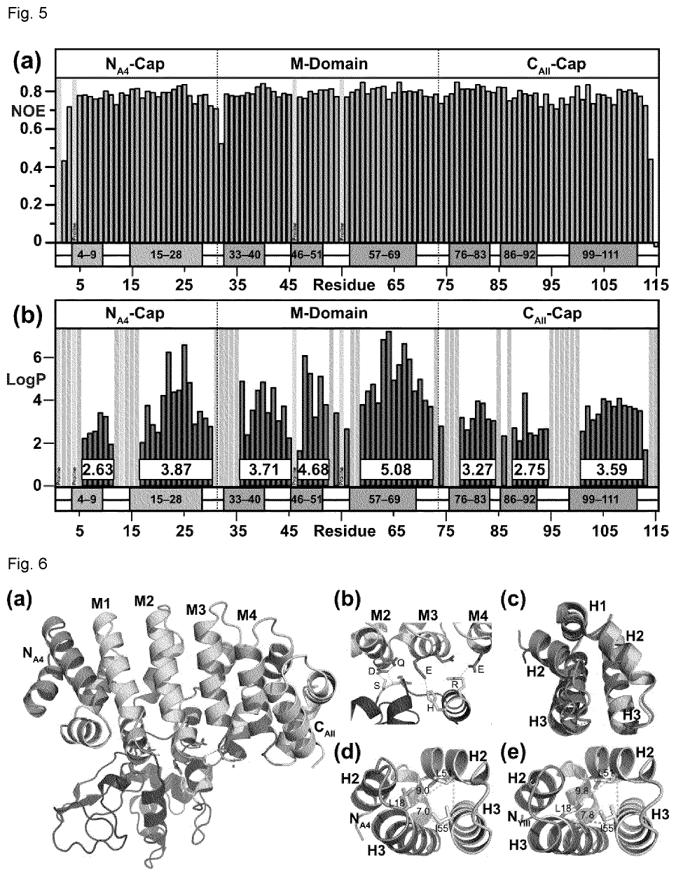

FIG. 5 shows conformational amide bond mobility and hydrogen exchange analysis for N_{A4}MC_{AII} at PH 5.5. (a) Heteronuclear 2D ¹⁵N{¹H}-NOE values determined for individual backbone amide bonds in N_{A4}MC_{AII} are plotted against the sequence. Colored boxes indicate helical segments in the N_{A4}-cap (blue), M module (orange) and C_{AII}-cap (green) as determined from the secondary shift analysis. (b) Logarithm of protection factors (log P) obtained from the hydrogen exchange analysis of individual residues in NA4MCAII plotted against the sequence. Grey bars indicate residues that exchange too fast to provide measurable P values while yellow bars indicate Proline residues or residues with overlapping amide resonances for which no P values could be obtained. Numbers in white boxes on red bars indicate averaged log P values for particular structural elements. All measurements were recorded at 20° C. on a 600 MHz spectrometer using 100 M N_{A4}MC_{AII} in 20 mM sodium phosphate at pH 5.5, containing 50 mM sodium chloride.

FIG. 6 shows crystal structure of N_{A4}M₄C_{AII} shows improved helical packing in N_{A4}-cap against internal repeat (a) Crystal structure of N_{A4}M₄C_{AII} determined in complex with lysozyme (PDB ID: 7QNP). The N_{A4}-cap, internal M modules and C_{AII}-cap are color-coded orange, green and yellow, respectively, while lysozyme is shown in blue. (b) Close-up of the contacts observed between N_{A4}M₄C_{AII} and lysozyme. Important residues are indicated as single letter amino acid codes. (c) Superposition of N-caps and first internal M modules from the crystal structure of N_{A4}M₄C_{AII}, shown in orange and green, and the crystal structure of N_{YIII}M₅C_{AII} (PDB: 5AEI) shown in magenta. (d,e) Distances between L18 in helix H3 of the N-cap to L51 in helix H2 and 159 in helix H3 of the first internal M module are indicated for (d) N_{A4}M₄C_{AII} and (e) N_{YIII}M₅C_{AII} (PDB ID: 5AEI).

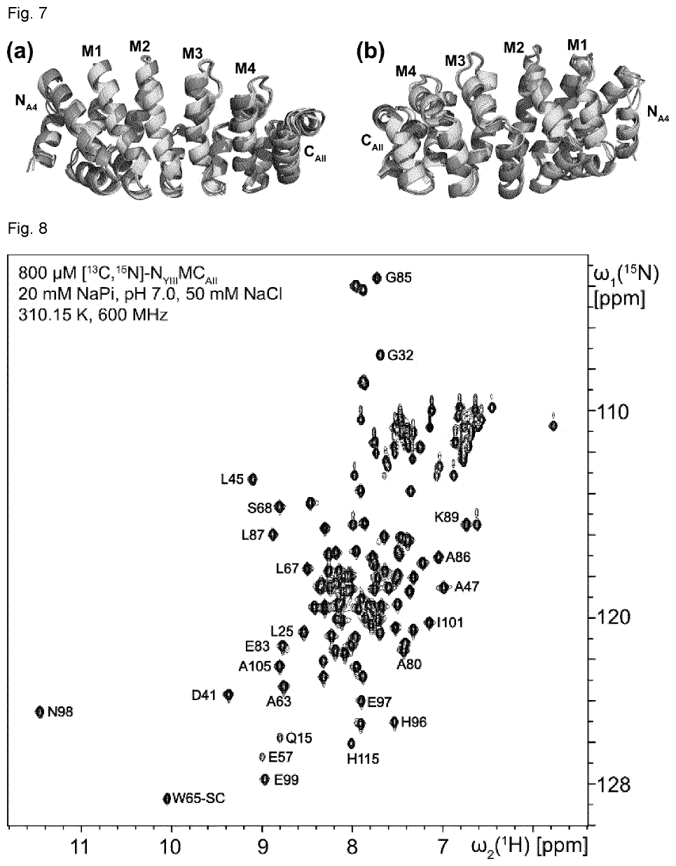

FIG. 7 shows PCS-derived solution structures of N_{A4}M₄C_{AI}. (a) Front and (b) back view of a superposition of three PCS-derived NMR solution structures derived from different starting models. All N_{A4}M₄C_{AII} solution structures reveal N_{A4}-cap conformations which are closely packed against the internal M module.

FIG. 8 shows 2D [¹⁵N,¹H]-HSQC spectrum of [¹³C,¹⁵N]-N_{YIII}MC_{AII} indicates a unique and well-folded population. The data were recorded at 37° C. on a 600 MHz spectrometer using 800 μM dArmRP in 20 mM sodium phosphate at pH 7 containing 50 mM sodium chloride.

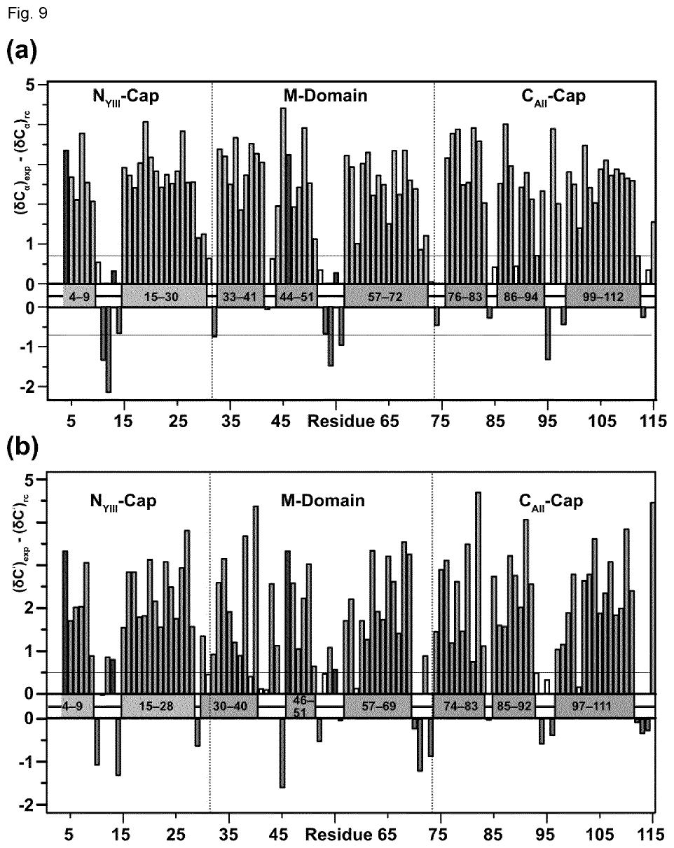

FIG. 9 shows secondary structure of N_{YIII}MC_{AII} from chemical shift indices. Secondary chemical shifts derived from assigned Cα (a) and C′ (b) spins of N_{YIII}MC_{AII}. Red bars indicate residues with secondary shift values that oppose α-helix formation while blue bars indicate proline residues. The lines at ordinate values of 0.7 (a) or 0.5 (b) indicate thresholds to define helical residues from Cα and C′ chemical shifts, respectively. Segments forming regular α-helices are schematically shown as colored boxes.

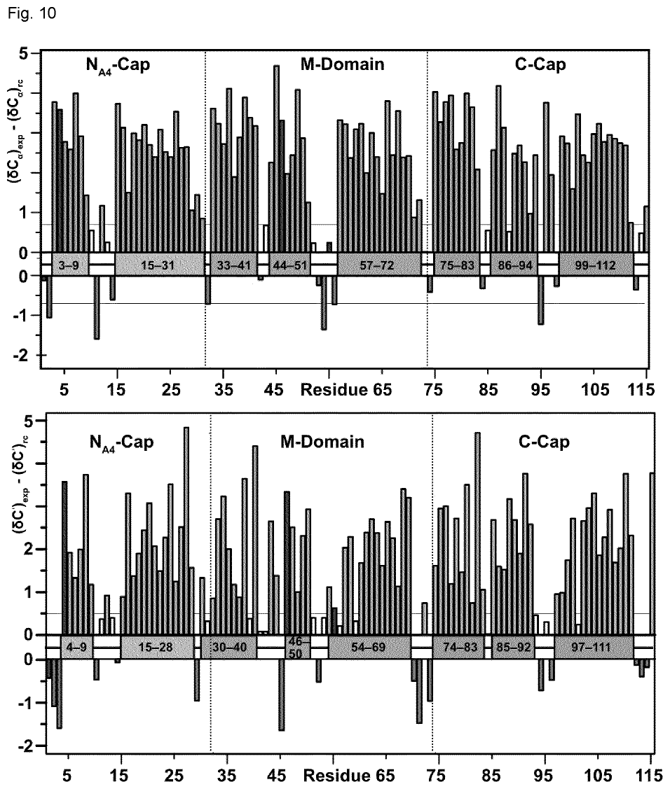

FIG. 10 shows secondary structure of N_{A4}MC_{AII} from chemical shift indices. Secondary chemical shifts derived from assigned Cα (a) and C′ (b) spins of N_{A4}MC_{AII}. Red bars indicate residues with secondary shift values that oppose α-helix formation while blue bars indicate proline residues. The lines at ordinate values of 0.7 (a) or 0.5 (b) indicate thresholds to define helical residues from Cα and C′ chemical shifts, respectively. Segments forming regular α-helices are schematically shown as colored boxes.

FIG. 11 shows [¹⁵N,¹H]-HSQC spectra of 100 μM N_{YIII}MC_{AII} in PBS buffer at pH 7 recorded at day 0 (a) and at day 64 (b) after incubation at 37° C. Both spectra were recorded at 37° C. and 600 MHz using identical measurement and processing parameters.

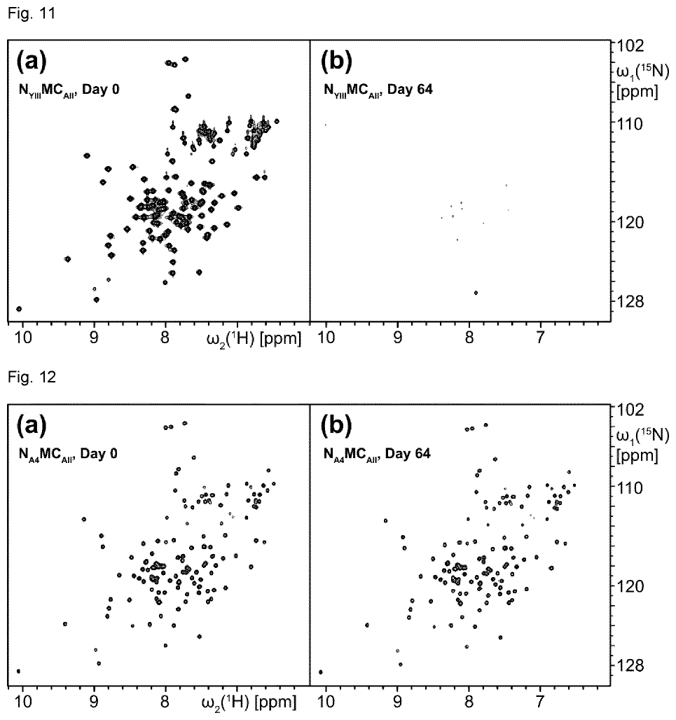

FIG. 12 shows [¹⁵N,¹H]-HSQC spectra of 100 μM N_{A4}MC_{AII} in PBS buffer at pH 7 recorded at day 0 (a) and at day 64 (b) after incubation at 37° C. Both spectra were recorded at 37° C. and 600 MHz using identical measurement and processing parameters.

Tab. 1 shows designed N-cap sequences and Rosetta energies of the corresponding NMC constructs. The sequences of this table relate to the SEQ ID Nos: 17-32 of the appending ST26 sequence protocol.
Tab. 2 shows cloning of target genes and expression plasmids.
Tab. 3 shows oligonucleotide primers used in this study. The sequences in this table relate to the SEQ ID Nos: 33-54 of the appending ST26 sequence protocol.
Tab. 4 shows data collection and refinement statistics of N_{A4}M₄C_{AII}:lysozyme.
Tab. 5 shows computational stability scanning mutagenesis of individual NH23-cap residues in N_{H23}MC_{AII} using the Rosetta software suite. Rosetta energy unit (REU) differences in NMC proteins resulting from single mutations after energy minimization are shown.
Tab. 6 shows Rosetta energy differences at individual N_{YII}- and N_{A4}-cap positions. Bold lines indicate positions with particularly large favorable REU differences.
Tab. 7 shows affinities of NM₄C proteins to (KR)₅-peptides.

### EXAMPLES

Designed Armadillo repeat proteins provide a promising scaffold for the engineering of modular sequence-specific peptide-binding proteins. In this context, “peptide” refers to the recognition sequence of a linear epitope. For such applications, dArmRP scaffolds need to provide exceptionally high stability and solubility to compensate for potentially unfavorable structural changes that can be a consequence of introducing and modifying various binding pockets in the internal modules. To further enhance the overall stability of dArmRPs, the inventors aimed at optimizing the N-capping repeat, using a combination of consensus and computational protein design. The inventors were motivated to focus on the N-capping repeat from a variety of observations summarized below.

### Example 1: NMR Analysis Reveals N_{YIII-Cap} Instability

NMR spectroscopy is a powerful method for the structural analysis of biomolecules in solution at atomic resolution, which the inventors intended to use in order to study the structural and dynamic adaptations of dArmRPs upon binding to their cognate target peptides. The initial isotope-labeled dArmRP prepared for NMR analysis comprised four internal M modules with the N_{YII}-cap and C_{AII}-cap as N- and C-terminal capping repeats, respectively. SDS-PAGE analysis of the purified dArmRPs revealed high purity and absence of undesired protein bands (data not shown). However, 2D [¹⁵N,¹H]-NMR spectra of the dArmRP showed a gradual appearance of a subset of new signals with low dispersion after several days at 37° C., suggesting partial sample degradation (FIG. 2a).

The inventors speculated that minute amounts of TEV protease, which was used to proteolytically remove the N-terminal (His)₆-tagged GB1 fusion domain during purification, might have remained in the NMR sample and exerted off-target cleavage that caused partial degradation of the dArmRP. To further investigate this, the inventors supplied a freshly prepared dArmRP NMR sample with 20 μg of TEV protease and compared the NMR spectra recorded at different time points with those from dArmRP samples without added TEV protease. Unexpectedly, the addition of TEV protease prevented sample degradation and the appearance of new peaks, which the inventors attributed to the protective effect of a storage buffer component such as EDTA, rather than to the TEV protease itself. Indeed, supplementing the NMR samples with 0.25 mM EDTA effectively prevented the appearance of additional peaks and protected the protein from degradation (FIG. 2b). This protective effect exerted by EDTA suggested the presence of catalytic amounts of a co-purifying metalloprotease from the E. coli expression host, which was not detectable by SDS-PAGE. Mass analysis of the partially degraded, [¹⁵N]-labeled NMR sample revealed a second protein species with a mass difference of 3105 Da to the intact dArmRP, which is in perfect agreement with proteolytic cleavage occurring between residues Q27 and 128, located in helix H3 of the N_{YIII}-cap. A subsequent bioinformatics search for known E. coli proteases that could potentially recognize this cleavage site provided no unambiguous results.

### Example 2: Protein Dynamics Suggest a Predominantly Well-Folded and Rigid N_{YIII}-Cap

The available crystal structures of dArmRPs containing the N_{YII}-cap indicate formation of two helices, H2 and H3, in the N_{YIII}-cap. However, proteolytic cleavage requires transient unfolding of helix H3 to provide access of the protease to the backbone of its recognized target site. To assess the conformational dynamics of the N_{YII}-cap at atomic resolution by NMR, the inventors prepared a minimalistic N_{YIII}MC_{AII} dArmRP containing only one internal M module (and thus termed NMC construct), flanked by the N_{YII}-cap and C_{AII}-cap. 2D [¹⁵N,¹H]-HSQC spectra of this construct revealed well-dispersed amide signals without apparent line-broadening, suggesting a uniform, well-folded protein population without conformational exchange in the μs- to ms-timescale (FIG. 8). Peak broadening of the backbone amide resonances was only observed for residues N33 and E34 of the internal M module and of N75 and E76 of the C-cap, indicating conformational dynamics in the intermediate exchange time regime for residues that constitute the beginning of helix 1. The assignment of the N_{YIII}MC_{AII} backbone resonances [BMRB accession number 51239] further provided the basis for a secondary structure analysis using the measured ¹³C_{α} and ¹³C′ chemical shift deviations from random coil (FIG. 9). The secondary ¹³C_{α} chemical shifts suggest that helix H2 in the N-cap is comprised of residues P4 to Q9 and helix H3 of residues Q15 to S30 (FIG. 9a). The secondary ¹³C′ chemical shifts confirm helical segments for residues P4 to Q9 in helix H2 and of residues Q15 to Q28 in helix H3 (FIG. 9b). A comparison of helices H2 and H3 of the N_{YIII}-cap in solution with those observed in crystal structures reveals identical secondary structure boundaries and thus confirms that the putative proteolytic cleavage site between Q27 and 128 is located within a helix.

To investigate amide bond mobilities in the pico- to nanosecond timescale within the N_{YIII}-cap, the inventors carried out 2D [¹H-¹⁵N]-heteronuclear NOE (HetNOE) experiments. The data analysis revealed near-maximal positive [¹H-¹⁵N]-HetNOEs and therefore restricts amide bond motions for most residues within the N_{YIII}-cap, the internal M module and the C_{AII}-cap (FIG. 3a). A slight decrease of the HetNOE, which corresponds to amide bond motions slightly faster than the overall tumbling of the protein, was observed for residues G31 and G32, which connect the N_{YIII}-cap to the internal M module, and for the C-terminus of the protein (FIG. 3a). In contrast, no significant increase in the backbone conformational dynamics was observed for the corresponding residues G73 and G74 that connect the M module with the C_{AII}-cap. Even though the mobilities of residues G31 and G32 are only slightly increased compared to the overall tumbling of the protein, the close vicinity to the proteolytic cleavage site Q27/128 may hint at a potential correlation between the increased linker mobility and transient initiation of helix H3 unfolding from the C-terminal end of the N-cap. However, the presented NMR data of N_{YIII}MC_{AII} shows a single NMR-observable protein population with an N-cap comprised of two stable helices and does not indicate conformational dynamics directly attributable to helix unfolding within the N_{YII}-cap.

### Example 3: Hydrogen Exchange Reveals Otherwise Invisible Transient Unfolded States

The aforementioned NMR analysis did not reveal detectable populations of alternative conformations and suggested formation of stable α-helices in the observable population of the N_{YIII}-cap. This implies that a conformation of N_{YIII}MC_{AII} where helix H3 of the N_{YIII}-cap is unfolded and accessible to proteolytic degradation must be so sparsely populated that it remains invisible to standard NMR analysis. To illuminate such marginally populated “invisible” states which are in dynamic equilibrium with the native state of N_{YIII}MC_{AII}, the inventors decided to analyze the amide proton hydrogen exchange (HX) with NMR to reveal the possible existence and relative populations of these states at single-residue resolution. Hydrogen exchange between water and protein amides directly correlates with the physical access of water molecules to individual amides in the protein, and the observed exchange rates k_{obs} can be described by equation 4:

$${k}_{o b s} = {k}_{int} \times {k}_{1} / {k}_{2}$$

where kᵢₙₜ is the residue-specific intrinsic exchange rate of a particular solvent-exposed amide proton, k₁ is the rate constant for the conversion from a solvent-protected (closed) into a solvent-exposed (open) state and k₂ is the rate constant for the reverse process. The closing equilibrium constant is referred to as protection factor P and is defined as the ratio of kᵢₙₜ/k_{obs}. Amide protons engaged in hydrogen bond networks such as in α-helices and those buried in the hydrophobic core of a protein typically reach high P values. An increased transient unfolding of helices H2 and H3 in the N-cap should therefore be reflected in small P values compared to the more compact parts of the protein.

The HX data of N_{YIII}MC_{AII} recorded at pH 5.5 revealed that the first 20 residues of the N-cap exchange too fast to be captured in the inventors' experimental setup, indicating that P values for these residues must be smaller than ca. 100 and that they spend at least 1% of the time in an open conformation (FIG. 3b). The only residues of the N-cap showing sufficient protection to be measurable comprised residues A21-A29 located within helix H3. The averaged log P value of ca. 2.46 for this segment corresponds to 0.3% of the time spent in an open conformation. Residues S30 to Q35, which comprise the linker between H3 of the N_{YIII}-cap and the beginning of H1 of the M module, were also exchanging too fast to be observable. However, residues 136 to A47, which constitute the majority of helix H1 of the internal M repeat up to the beginning of helix H2, exchange with an averaged log P value of 2.49, which closely resembles the value of the segment comprising residues A21-A29, suggesting that these segments unfold together as a cooperative unit (FIG. 3b). Residues L48-L52 of helix H2 and residues I59-S72 of helix H3 in the M module show similar log P values of 4.1 and 4.04 that correspond to ca. 0.005% and 0.003% of the time spend in an open conformation, respectively (FIG. 3b). The similar log P values for H2 and H3 suggest that these helices also unfold in a cooperative manner. The helices in the C-cap show more similar log P values amongst themselves, with values of 2.92, 2.56 and 3.19 for residues K78-A84 in helix H1, K89-Q94 in helix H2 and I101-L112 in helix H3, respectively (FIG. 3b).

The HX data convincingly show that the residues in the N_{YIII}-cap have the lowest protection factors and that they spend at least 0.3% of the time in an open conformation, which enables proteases to access the polypeptide chain. Helix 2 of the internal M module appears weakly protected and unfolds cooperatively with H3 of the N_{YIII}-cap; however, the cooperatively unfolding helices H2 and H3 of the M module possess ca. 50-75-fold higher protection than helix H1, which can be rationalized by the more protected environment provided by packing against helices H2 and H3 of both N- and C-caps. The corresponding P values of the C-cap are severalfold increased compared to the N-cap, which implies a better overall packing of the C-cap and suggests that the stability of the N-cap could possibly be improved by optimization of the repeat packing.

### Example 4: Computational N-Cap Design for Enhanced Stability

The HX experiments mentioned above have revealed that the N-cap spends a small but significant amount of time in an “open” conformation that gives access to the amide protons, while the M module shows enhanced protection and stability. Previous experiments have further shown that helices H2 and H3 of the M module can substitute the N-cap in dArmRPs without significant losses in stability or solubility. Due to these favorable properties, the inventors decided to use an N_{H23}-cap composed of helices H2 and H3 of the M module as a starting template for a new N-cap, in combination with one internal M module and a C_{AII}-cap, for an in-silico design of a new N-cap using the Rosetta macromolecular modeling program.

A scanning mutagenesis screen probing each individual position in the N_{H23}-cap showed that the largest energetic gains in Rosetta can be obtained by mutation of surface-exposed residues located in helices H2 and H3 (Tab. 5), suggesting that the packing and energy of the existing hydrophobic core, transferred from the M module, is scored favorably by Rosetta. Due to this finding, the inventors' design strategies included simultaneous optimization of either all surface-exposed or all residues of the N_{H23}-cap, using a combination of the Rosetta fixbb and relax protocols. Rosetta-proposed mutations occurred mainly for surface-exposed residues, confirming the initial results of the scanning mutagenesis screen (Tab. 1). The total Rosetta energy units (REUs) of the newly designed NMC variants after energy minimization ranged from ca. 350-358 REUs, which compares favorably to the 333 and 335 REUs obtained for the constructs containing the original N_{YIII}-cap and the template N_{H23}-cap, respectively (Tab. 1).

The N-cap variant A6, a hybrid construct composed of the original helix H2 from the starting template N_{H23} and a newly designed helix H3, scored 17 REUs better than the original N_{YIII}MC, whereas all variants containing both newly designed helices H2 and H3 scored at least 24 REUs better than N_{YIII}MC. This indicates that the REU gains were more than twofold larger in helix H3 compared to helix H2. All N-cap variants with optimized helices H2 and H3 differ by less than 1.7 REUs from each other and show only few conservative sequence variations (Tab. 1). The sequence composition of the newly designed N-caps shows a large proportion of charged amino acids, which account for about one third of all residues, and an even slightly larger proportion of the helix-forming residues Leu and Ala. Interestingly, all seven Gln residues in the original N_{YIII}-cap sequence have been replaced to either Lys, Glu or Leu in the new N-cap sequences by Rosetta.

A REU comparison of each residue in the original N_{YIII}-cap with the corresponding residue in the highest-scoring N_{A4}-cap reveal that five mutations M6L, Q9L, Q19L, K24A and S26A, which are located at or in the hydrophobic core, account for a gain of 18.7 REUs. Most surface-exposed residues show smaller individual REU gains but contribute favorably to the overall stability of the new N_{A4}-cap in Rosetta (Tab. 6). This suggests that transfer of the hydrophobic core from an internal M module obtained from consensus design to the N-cap provided mainly stability, while redesign of surface-exposed residues addressed both protein solubility and stability.

### Example 5: Experimental Stability Assessment of N-Cap Designs

To experimentally assess the stability of the newly designed N-caps, the inventors expressed and purified the corresponding NMC constructs to analyze both denaturant-induced equilibrium unfolding and thermal unfolding of these proteins by circular dichroism (CD) spectroscopy. Denaturant-induced equilibrium unfolding of the NMC constructs was achieved with increasing concentrations of guanidine hydrochloride (GdnHCl) in PBS buffer at pH 7 and was monitored by recording the CD signal at 222 nm. The denaturation midpoint concentrations Dₘ, which indicate the GdnHCl concentration required to unfold 50% of the total protein, were derived from a nonlinear fit of the sigmoidal unfolding curves using a Boltzmann function (FIG. 4). The analysis showed cooperative unfolding for all tested constructs and provided Dₘ values of 1.86 and 2.29 M GdnHCl for N_{YIII}MC and N_{H23}MC, respectively, while all NMC constructs containing a newly designed N-cap showed Dₘ values ranging from 3.12 M GdnHCl for N_{A6}MC to 3.61 M GdnHCl for N_{A4}MC (FIG. 4).

The calculated Rosetta energies agree remarkably well with the ranking of experimentally determined stabilities towards denaturant-induced unfolding and indicate a correlation of one REU for a change in Dₘ of roughly 0.06 M GdnHCl. The optimization of surface-exposed residues appears to be a very important contributor to the large overall stability enhancement since the sole transfer of helices H2 and H3 of an internal M module, which provided the stable hydrophobic core, into the N_{H23}-cap increased the Dₘ value only to 2.29 M GdnHCl. N-caps obtained after including redesign of surface-exposed residues all showed Dₘ values above 3 M GdnHCl. The large increase in Dₘ from 1.86 M for N_{YIII}MC to 3.61 M GdnHCl in N_{A4}MC underlines the significantly improved stability of the novel N-caps and is about five times larger than all combined Dₘ gains from previous N-cap engineering efforts.

To complement and support the denaturant-induced unfolding data, the inventors followed thermal unfolding of the NMC constructs by recording the CD signal at 222 nm during a slow and steady temperature increase of 1° C. per minute from 25 to 95° C. The resulting sigmoidal thermal unfolding curves were fit using a nonlinear Boltzmann function (FIG. 4), and the thermal melting temperatures Tₘ were obtained from the second derivative of the fitted curve, which equals to zero at Tₘ. In contrast to the denaturant-induced unfolding data, the thermal unfolding stabilities did not follow the exact ranking suggested from the Rosetta energies (FIG. 4); however, all NMC constructs containing newly designed N-caps showed significantly elevated Tₘs between 87.1 and 91.5° C., compared to Tₘs of 75.9 and 74.8° C. for N_{YIII}MC and N_{H23}MC, respectively, and thus confirmed the high stability of the new N-caps observed in denaturant-induced unfolding. Furthermore, all NMC constructs showed completely cooperative and reversible thermal unfolding (data not shown).

### Example 6: NMR Analysis of N_{A4}MC

The large increase in stability for the N_{A4}MC construct prompted the inventors to further characterize the structural and dynamic properties of this protein by NMR spectroscopy. The inventors therefore prepared ¹³C,¹⁵N-labeled N_{A4}MC to assign the backbone resonances (BMRB accession code 51240) and to derive secondary shifts, which indicated no significant differences in the helical properties of the two proteins N_{YIII}MC and N_{A4}MC (FIG. 10). Furthermore, heteronuclear NOE data showed no increased conformational mobilities for the backbone amides in the N_{A4}MC protein, including the newly designed N-cap (FIG. 5), which indicates a rigid conformation of the predominant population, comparable to the data of the N_{YIII}MC protein.

The inventors then analyzed and compared the long-term stabilities of the new N_{A4}MC protein and the N_{YIII}MC protein. In contrast to the previously observed slow degradation of the N_{YIII}M₄C protein, presumably by co-purified traces of an E. coli metalloprotease, the smaller N_{YIII}MC construct appears to completely precipitate with prolonged incubation at 37° C. (FIG. 11), which is likely due to a reduced solubility of the populations with partially unfolded helices and/or repeats in the smaller protein, compared to the proteins containing four internal modules. The N_{A4}MC protein with the newly designed N-cap, on the other hand, does not show any changes in the pattern or intensity of the amide resonances after 64 days (FIG. 12), indicating that the novel N_{A4}-cap completely prevents adverse sample modifications, such as proteolysis, and aggregation and confirms the increased stability seen in the unfolding experiments.

### Example 7: Hydrogen Exchange of N_{A4}-Cap Indicates Stabilized Folding Units

The previous HX data of the N_{YIII}MC construct showed that the N_{YIII}-cap is the least stable repeat, and it spends at least 0.3% of the time in an open conformation, which provides a rationale for the observed sample instability. To compare these properties with those of the new N-cap in the N_{A4}MC protein, the inventors analyzed the amide HX in the N_{A4}MC protein using the identical setup as for N_{YIII}MC (FIG. 5). The previously unobservable H2 of the N_{YIII}-cap is sufficiently stabilized in the N_{A4}-cap to provide measurable exchange rate constants, which indicate a log P of 2.63 for residues L6 to K11, showing that H2 spends 0.23% of the time in an open conformation. The linker segment comprising residues S12-E16 exchanged too fast to be observable; however, residues 117-S30 showed a significantly increased log P of 3.87, which corresponds to only 0.014% of the time in an open conformation.

The only observable segment in the N_{YIII}-cap, which appears to contain the proteolytic target cleavage site in the N_{YIII}M₄C protein, comprised residues A21-A29 with a log P of 2.46 (FIG. 3). In the N_{A4}-cap, the corresponding segment now shows a log P of 4.47, increased by more than two orders of magnitude, which allows the inventors to rationalize the increased sample stability (FIG. 5). Moreover, the internal M module shows more than a 15-fold increase in P values for helix H1, about a 4-fold increase for helix H2 and about a 10-fold increase for helix H3 compared to the P values obtained in the N_{YIII}MC construct. Albeit weakly, this stability increase is even further propagated into the C-cap where helices H1, H2 and H3 show P value improvements of more than 2-fold, 1.5-fold, and 2.5-fold, respectively. This indicates that the improved stability and tight packing of the N_{A4}-cap against the internal module provides stability benefits within the entire protein.

### Example 8: Crystal Structure of Nₐ₄M₄C Highlights Tighter N-Cap Packing

To gain insight into the structural details of the novel N_{A4}-cap, the inventors solved the crystal structure of N_{A4}M₄C, which was accidentally co-purified and co-crystalized with lysozyme, at 1.59 Å resolution (PDB ID: 7QNP). The binding interface between the dArmRP and lysozyme involves mainly polar interactions between residues on helices H1 in modules M₂, M₃ and M₄ of the dArmRP and residues in lysozyme (FIG. 6). Affinity measurements between N_{A4}M₄C and lysozyme by isothermal titration calorimetry indicate a very weak interaction with a K_{d} of about 6.6 μM (data not shown).

The helical boundaries observed in the crystal structure correspond well with the secondary shifts determined by NMR. This confirms that helices H2 and H3 of the N_{A4}-cap are comprised of residues L3-K11 and E15-S28, respectively. A structural comparison between the N_{A4}- and N_{YIII}-caps shows that helix H3 of the N_{A4}-cap packs more closely against helices H2 and H3 of the first M module (FIG. 6), which further supports the increased protection factors for helices located in both the N_{A4}-cap and the neighboring M module. For example, the C_{α}-C_{α} distances from L18, which is a common residue in both N_{A4}- and N_{YIII}-caps, to L51 in helix H2 and 159 in helix H3 of the M module, decreases from 9.8 to 9.0 and 7.8 to 7.0 Å, respectively (FIG. 6). Other available crystal structures of dArmRPs containing the N_{YIII}-cap (PDB: 5MFH, 4V30, 5MFD) show values of 10.7-11 Å and 8.4-9.1 Å for the corresponding distances between L18-L51 and L18-I59, respectively.

### Example 9: Novel N-Caps do not Impact Target Peptide Binding

dArmRP are modular peptide-binding molecules that interact with their cognate target peptides via specific interactions mediated by the internal M modules. The capping repeats provide stability and solubility and do not contribute to the specific target peptide recognition. To assess the non-binding properties of the novel N-caps, the inventors determined the binding affinity of dArmRPs, containing either the novel N-caps or the original N_{YIII}-cap, four internal M repeats and the C_{AII}-cap, towards the (KR)₅-peptide. The obtained results show similar K_{d}'s between 22-49 nM for all tested combinations. In particular, the constructs with the well-characterized N_{A4}- and N_{YIII}-caps yield K_{d}'s of 30.5±2.3 nM and 36.1±2.9 nM, respectively. This suggests that the novel caps do not significantly impact peptide binding, which is one of the desired features of N-caps.

### Example 10: Solution Structure of N_{A4}M₄C_{AII}

Previous NMR studies of dArmRPs containing the N_{YIII}-cap proved to be difficult due to the low stability of the N-cap. The recent NMR structure calculation of N_{YIII}M₄C revealed once more that the low stability of the N_{YIII}-cap resulted in multiple solutions in the structure calculation, containing contributions from a rather extreme detachment of fluctuating N_{YIII}-caps from the first internal M module, creating a rather unrealistic description of the N_{YIII}-cap conformation. As a first application of the new N_{A4}-cap and to assess whether the new N_{A4}-cap facilitates NMR studies, the inventors determined the solution structure of the N_{A4}M₄C_{AII} protein using a combination of NOE- and PCS-derived distance constraints. The obtained set of three N_{A4}M₄C_{AII} solution structures superimpose with an RMSD of 0.39±0.24 Å, indicating good convergence in the structure calculation, and with an RMSD of 1.63 Å to the N_{A4}M₄C_{AII} crystal structure. In stark contrast to the solution structure of N_{YIII}M₄C, the PCS-refined structure calculation of the N_{A4}M₄C_{AII} protein provides conformations where the N_{A4}-cap is firmly packed against the M module (FIG. 7). Large conformational fluctuations of the N_{A4}-cap are absent, which further highlights the improved stability and overall properties of the novel N_{A4}-cap that will facilitate biochemical and structural investigations of dArmRPs in solution.

### DISCUSSION

The inventors describe here the stabilization of the N-capping repeat of dArmRPs by employing a combination of consensus and computational protein design. The original N_{YIII} was shown to be susceptible to aggregation and degradation, even though NMR analysis of the N_{YIII}-cap did not show any obvious indications for an unstable capping repeat. However, hydrogen exchange experiments revealed a very low but significant population of unfolded helices in the N_{YIII}-cap, which provide the molecular basis for aggregation and degradation. The inventors decided to employ a previously engineered internal M module, obtained from consensus design, as structural template for a computational optimization using the Rosetta software. Most residues within the hydrophobic core did not to require optimization, but the vast majority of surface-exposed residues were optimized during in silico design. This optimization resulted in very large stability improvements in GdnHCl-induced equilibrium unfolding, which were up to five-fold larger than all gains combined from previous engineering efforts. The inventors could furthermore demonstrate that these novel N-caps show more than a 100-fold reduction in the populations of unfolded states, which provides the basis for the elimination of the previously observed aggregation and degradation propensities. The determined crystal structure of the N_{A4}M₄C_{AII} protein indicated tighter packing of the novel N-cap to the first internal module, which provided structural evidence for the improved stability of dArmRPs containing the new N-cap. As a first application, the inventors used the new N-cap to solve the solution structure of N_{A4}M₄C_{AII}, which, in contrast to the previously determined solution structure of N_{YIII}M₄C_{AII}, shows good convergence and a well-packed N_{A4}-cap. This work clearly demonstrates that combining consensus and computational protein design is a very powerful approach for improving protein stability.

### Material and Methods

### Cloning of Target Genes

All genes encoding dArmRPs were PCR-amplified from a codon-optimized N_{YIII}M₃C_{AII} gene using the oligonucleotide primer and template DNA combinations listed in Tab. 2 and 3. PCR products encoding dArmRPs with one internal module were cloned into the expression vector pEM3BT2 using the SapI/BamHI restriction sites. Genes encoding dArmRPs with four internal modules were assembled by ligation of a 5′- and a 3′-PCR product, separately digested with XbaI/SapI and SapI/BamHI, respectively, into XbaI/BamHI-digested pEM3BT2. All constructs were cloned as fusion constructs to an N-terminal (His)₆-tagged GB1 domain, which is separated with a flexible linker encoding a TEV-protease cleavage site for facile proteolytic removal of the N-terminal (His)₆-GB1. The expression plasmid pEM3BTC, which encodes a HRV 3C-protease cleavage site in the linker between (His)₆-GB1 and the target gene, was generated by mutagenesis PCR of the pEM3BT2 plasmi using the 3BTC_Fwd and 3BTC_Rev oligonucleotide primers. The MNG-3BTC plasmid for expression of target peptides fused to mNeonGreen was prepared by ligation of the SapI/BgIII-digested PCR product encoding mNeonGreen into SapI/BamHI-digested pEM3BTC. Complementary oligonucleotides encoding the (KR)₅-target peptide were annealed after heating to 95° C. by passive cooling to 25° C. and were subsequently introduced into MNG-3BTC using the BamHI/BsaI restriction sites. The single Cys-variants E16C, Q93C and S222C of N_{A4}MC_{AII}, required for the site-specific attachment of dia- and paramagnetic tags, were prepared by mutagenesis as previously described.

### Protein Expression and Purification

All proteins were expressed in E. coli BL21-Gold (DE3) cells (Agilent Technologies) growing at 37° C. with shaking in 200 ml 2YT medium. Expression was induced with 1 mM IPTG at an OD₆₀₀ of ca. 0.6-0.8 for ca. 16 h at 30° C. [¹³C,¹⁵N]-labeled proteins for NMR analysis were also expressed using E. coli BL21-Gold (DE3) cells but grown in minimal medium. After harvesting by centrifugation, the obtained cell pellets were resuspended in 15 ml buffer A (50 mM sodium phosphate at pH 7.7, 500 mM sodium chloride, 20 mM imidazole, 30 μM sodium azide) supplemented with 5 mM magnesium sulfate, 1 mg/ml hen egg white lysozyme (Sigma-Aldrich) and 0.05 mg/ml DNaseI (Roche). Cells were lysed with a Branson Ultrasonics 250 Sonifier (Branson Ultrasonics) for 3 min on ice using a duty cycle of 70% and an output power of 4. Insoluble debris was subsequently removed by centrifugation and the supernatant was filtered through a 0.2 μm sterile syringe filter unit (Sartorius) before purification on a 5 ml HisTrap HP column as previously described. The N-terminal (His)₆-GB1 fusion was then removed by proteolytic cleavage with 2 mg TEV protease in case of dArmRPs and with 1 mg HRV 3C protease for the (KR)₅-mNeonGreen fusion. After separation of the target protein from (His)₆-tagged species by re-application on a 5 ml His Trap HP column (GE Healthcare), the purified proteins were dialyzed against NMR buffer (20 mM sodium phosphate, 50 mM sodium chloride, 30 μM sodium azide) and concentrated in 3 kDa MWCO ultrafiltration devices (Merck Millipore). Proteins intended for affinity measurements by fluorescence anisotropy were dialyzed against PBS (50 mM sodium phosphate at pH 7.4, 150 mM sodium chloride, 30 μM sodium azide). The N_{A4}M₄C_{AII} construct prepared for crystallization was additionally purified by size exclusion chromatography on a HiLoad 26/60 Superdex 75 column (GE Healthcare) equilibrated in 10 mM Tris-HCl at pH 7.6 prior to concentration in a 10 kDa MWCO ultrafiltration device (Merck Millipore).

TEV protease was prepared as previously described (Michel, E., and Wüthrich, K. (2012), J. Biomol. NMR 53, 43-51). HRV 3C protease in pET24b was expressed in E. coli BL21-Gold (DE3) cells growing in 1 L 2YT medium with shaking at 25° C. Protein expression was induced at OD₆₀₀ of 0.6 with 0.5 mM IPTG for 16 h. Cells were harvested as described above and were resuspended in 40 ml buffer A-3C (40 mM HEPES-NaOH at pH 8, 300 mM sodium chloride, 20 mM imidazole, 1 mM DTT, 10% (v/v) glycerol) and lysed with a Branson Ultrasonics Sonifier 250 for 10 min on ice with a duty cycle of 30% and an output level of 4. Clearing of the sample was performed as described above and the filtered sample was applied on a 5 ml HisTrap HP column in buffer A-3C. After washing with 15 column volumes of buffer A-3C, the HRV 3C protease was eluted with a 100 ml linear gradient of buffer A-3C to buffer β-3C (same as buffer A-3C but containing 300 mM imidazole) and dialyzed overnight in a 12-14 kDa MWCO dialysis membrane (Spectrum Labs) at 4° C. against 2 L of buffer 3C (10 mM HEPES-NaOH at pH 8, 150 mM sodium chloride, 5 mM EDTA, 1 mM DTT, 10% (v/v) glycerol). The protein solution was then further supplemented with glycerol to a final concentration of 20% (v/v) glycerol, and aliquots containing 2 mg HRV 3C protease were flash-frozen in liquid nitrogen and stored at −80° C.

### NMR Analysis

NMR experiments were measured at 310.15 K on a Bruker Avance 600 spectrometer equipped with a cryogenic triple-resonance probe-head. All NMR samples were supplemented with 5% (v/v) D2O. Backbone resonances were assigned with 2D [¹⁵N,¹H]-HSQC, 3D HNCA, 3D HNCACB, 3D HNCO, 3D HN(CA)CO and 3D CBCA(CO)NH experiments (Sattler, M., et al., (1999), Prog. Nucl. Magn. Reson. Spectrosc. 34, 93-158). Secondary structure analysis was performed using the Cα and C′-shifts according to the chemical shift index protocol (Wishart, D. S., and Sykes, B. D. (1994), J. Biomol. NMR 4, 171-180). Backbone amide mobilities were determined from 2D ¹⁵N{1H}-NOE data recorded using a relaxation delay of 5 s (Kay, L. E., Torchia, D. A., and Bax, A. (1989), Biochemistry (Mosc). 28, 8972-8979).

The amide proton exchange experiments were performed at pH 5.5 using 0.1 mM protein in a total volume of 500 μl. Proton exchange was started by redissolving the lyophilized protein sample in 500 μl D₂O, followed by immediate and continued measurement of 2D [¹⁵N,¹H]-HSQC experiments after regular time intervals. All measurement and processing parameters were kept identical throughout the data acquisition series and the sample was kept constantly at 37° C. in between NMR measurements. The disappearance of individual amide resonances was followed by cross-peak integration using the software CARA (Keller, R. (2004), Cantina Verlag, Goldau, Switzerland.) and the residue-specific observed exchange rates k_{obs} were determined from a single exponential decay fit to the amide cross-peak intensity versus time. Protection factors P for individual residues were determined from the ratio of intrinsic and observed exchange rates kᵢₙ/k_{obs} (Damberger, F. F. et al., (2013), Proc. Natl. Acad. Sci. U.S.A 110, 18680-18685; Conway, P., et al., (2014), Protein Sci. 23, 47-55). The structure determination of N_{A4}M₄C_{AII} in solution using PCS-constraints was performed according to the recently described procedure (Cucuzza, et al., (2021), J. Biomol. NMR 75, 319-334.). Three tag-attachment sites E16C, Q93C and S222C were used for installation of dia- and paramagnetic tags. The initial structural models used as templates for the NMR structure calculation were derived from N_{YIII}M₅C_{AII} (PDB ID: 5AEI) by deletion of the N_{YIII}-cap and using the PyMOL mutagenesis wizard to convert the residues of the first M module into the corresponding N_{A4}-cap residues, from a Rosetta model obtained by energy minimization of this first structural model using the Relax protocol, and from the crystal structure of N_{A4}M₄C_{AII} determined in this work.

### Computational Protein Design

The structural model N_{YIII}MC_{AII} used for computational protein design in Rosetta was created by least squares superposition of the M modules of N_{YIII}M and MC_{AII} fragments, derived from the crystal structure of N_{YIII}M₅C_{AII} (PDB: 5AEI). All Rosetta calculations were performed using the Rosetta 3.9 release and the “beta_nov16” scoring function. Rosetta all-atom refinements of the initial N_{YIII}MC_{AII} structural model were obtained by running the Relax protocol to generate 10 refined structural models, each obtained from a total of 20 cycles of sidechain repack and minimization. The obtained refined structural models served as templates for computational protein design of the N-cap with the fixbb protocol (Kuhlman, B., et al., (2003) Design of a novel globular protein fold with atomic-level accuracy, Science 302, 1364-1368), which was run with 500 trajectories for each of the 20 output structures. N-cap residues chosen for sidechain-rotamer optimization by Rosetta were tested for all possible amino acids except cysteine (ALLAAxC, SEQ ID NO:55). Residues 1, 2, 4, 5, 8, 11-13, 15, 16, 19, 20, 23, 26 and 27 comprised the set of surface-exposed amino acids. The obtained designs were subjected to an all-atom refinement as described above and the average Rosetta energy was calculated for the 10 output structural models.

### Protein Stability Assessment by CD Spectroscopy

Denaturant-induced equilibrium unfolding and thermal unfolding experiments of the NMC constructs was monitored by CD spectroscopy on a Jasco J-715 instrument using a cylindrical cuvette with 1 mm pathlength equipped with temperature control. All measurements were performed using 15 μM protein in NMR buffer with a data pitch of 0.5 nm, scanning speed of 100 nm/min, response time of 4 s, bandwidth of 1 nm and a sensitivity of 100 mdeg. Denaturant-induced equilibrium unfolding was achieved by overnight incubation at room temperature with various concentrations of GdnHCl (Fluka) and measured via the ellipticity at 222 nm with 25 accumulations at 20° C. The fraction of unfolded dArmRP at each concentration of GdnHCl was calculated according to equation 1:

$${F}_{U} = \frac{{θ}_{N} - θ ( x )}{{θ}_{N} - {θ}_{U}}$$

with θ_{N} and θ_{U} indicating the mean residue ellipticities for fully native and fully unfolded protein, respectively, and θ(x) the observed ellipticity at x M GdnHCl. Denaturation midpoint concentrations Dₘ were then estimated from a nonlinear Boltzmann fit of the obtained sigmoidal unfolding curves according to equation 2:

$${f}_{U} ( x ) = \frac{{A}_{1} - {A}_{2}}{1 + {e}^{( x - x 0 ) / dx}} + {A}_{2}$$

where x is the concentration of GdnHCl in M, x₀ is Dₘ, and A₁ and A₂ are the baselines of the unfoldeded fraction for fully folded and unfolded protein of 0 and 1, respectively. Note that this formula only serves to estimate the transition midpoint and does not describe the folding equilibrium.

Thermal unfolding of the NMC constructs was achieved with a temperature increase of 1° C. per minute from 25 to 95° C. while recording the ellipticity at 222 nm. The resulting sigmoidal thermal unfolding curves were fit using a nonlinear Boltzmann function and the thermal melting temperatures Tₘ were obtained from the second derivative of the curve fit, which equals zero at Tₘ.

### Crystallization and Structure Determination

60 mg/ml of N_{A4}M₄C_{AII} in 10 mM Tris-HCl at pH 7.6 was applied to sparse-matrix screens from Molecular Dimensions and Hampton Research in 96-well plates (Corning) at 20° C. to identify crystallization conditions. Protein solutions were mixed at ratios of 1:1, 1:2 and 1:3 with reservoir solution to volumes of 300-400 nl and equilibrated against 30 μl reservoir solution in sitting-drop vapor diffusion experiments. Crystals obtained in 35% (v/v) dioxane were picked after addition of 30% (v/v) ethylene glycol as cryoprotectant and flash-frozen in liquid nitrogen. Diffraction data were collected with a Dectris Eiger X 16M detector on the X06SA beamline at the Swiss Light Source (Paul-Scherrer Institute, Villigen, Switzerland) and was processed using the programs XDS (Kabsch, W. (2010), Acta Crystallogr D Biol Crystallogr 66, 125-132), Aimless (Evans, P. R., and Murshudov, G. N. (2013), Acta Crystallogr D Biol Crystallogr 69, 1204-1214.) and MOLREP (Vagin, A., and Teplyakov, A. (2010), Acta Crystallogr D Biol Crystallogr 66, 22-25). The crystal structure was determined by molecular replacement with PDB 5aei, followed by structure refinement using the program REFMAC (Murshudov, G. N., et al., (1999), Acta Crystallogr D Biol Crystallogr 55, 247-255) and model building in COOT (Emsley, P., and Cowtan, K. (2004), Acta Crystallogr D Biol Crystallogr 60, 2126-2132). The R_{free} was calculated with five percent of separated data and PROCHECK (Laskowski, R. A., et al., (1993), J. Mol. Biol. 231, 1049-1067) was used to validate the final structure. All data collection and refinement statistics are shown in Tab. 4.

### Affinity Determination

Affinities of NM₄C_{AII} proteins with various N-caps to the (KR)₅ peptide fused to mNeonGreen were determined by fluorescence anisotropy on a Tecan Safire II plate reader equipped with a fluorescence polarization module. A fixed amount of 2 mM (KR)₅-sfGFP was titrated in four replicates with 24 dilutions ranging from 160 pM to 20 μM dArmRP. Excitation and emission wavelengths were set to 470 and 510 nm, respectively, using a bandwidth of 10 nm. The averages of four replicates were subtracted with the anisotropy obtained with the lowest dArmRP concentration and were fit, as previously described (Hansen, S., et al., (2016), J. Am. Chem. Soc. 138, 3526-3532.), to equation 3:

$${F}_{A P} ( {c}_{A} ) = \frac{m ( - {K}_{d} - {c}_{A} - {c}_{P} + \sqrt{{( {K}_{d} + {c}_{A} + {c}_{P} )}^{2} - 4 {c}_{A} {c}_{P} )}}{- 2 {c}_{P}}$$

where F_{AP} is the fraction of bound peptide, c_{A} is the concentration of dArmRP, cₚ is the fixed concentration of peptide, K_{d} is the dissociation constant and m is the anisotropy amplitude between unbound and bound peptide.

### Tables

TABLE 1

| N- | | SEQ ID | | SEQ ID | Rosetta Energy |
|---|---|---|---|---|---|
| Cap | Helix 2 | NO: | Helix 3 | NO: | (NMC) |
| A4 | PDLPKLVKLLKSS | 17 | NEEILLKALRALAEIAS | 25 | −358.3 |
| A5 | PDLPKLVKLLKSS | 18 | NEEILLKALKALAEIAS | 26 | −357 |
| A7 | PDLPKLVKLLKSS | 19 | DEETLLKALRILAEIAS | 27 | −356.9 |
| A9 | PDLPKLVKLLKSS | 20 | DEKTLLEALKTLAEIAS | 28 | −356.8 |
| A8 | PDLPKLVKLLKSS | 21 | DEETLLKALKTLAEIAS | 29 | −356.6 |
| A6 | GALPALVQLLSSP | 22 | DEETLLKALKTLAEIAS | 30 | −349.8 |
| H23 | GALPALVQLLSSP | 23 | NEQILQEALWALSNIAS | 31 | −335 |
| Y | GELPQMVQQLNSP | 24 | DQQELQSALRKLSQIAS | 32 | −332.9 |

TABLE 2

| Construct | | Template DNA | Recipient |
|---|---|---|---|
| name | Oligonucleotides | for PCR | Plasmid |
| NH₂₃MC_{AII} | H23MC_Fwd/ | N_{YIII}M₃C_{AII} | pEM3BT2 |
| | H23MC_Rev | | |
| N_{YIII}MC_{AII} | M3_Fwd/Y_Rev | N_{YIII}M₃CA_{II} | pEM3BT2 |
| N_{SH2}MC_{AII} | V1_Fwd/V1_Rev | N_{H23}MC_{AII} | pEM3BT2 |
| N_{A4}MC_{AII} | V41_Fwd/V41_Rev | N_{SH2}MC_{AII} | pEM3BT2 |
| N_{A5}MC_{AII} | V42_Fwd/V41_Rev | N_{SH2}MC_{AII} | pEM3BT2 |
| N_{A6}MC_{AII} | V5_Fwd/V5_Rev | N_{H23}MC_{AII} | pEM3BT2 |
| N_{A7}MC_{AII} | V6_Fwd/V6_Rev | N_{SH2}MC_{AII} | pEM3BT2 |
| N_{A8}MC_{AII} | V5_Fwd/V6_Rev | N_{SH2}MC_{AII} | pEM3BT2 |
| N_{A9}MC_{AII} | V5_Fwd/V8_Rev | N_{SH2}MC_{AII} | pEM3BT2 |
| N_{H23} M₄C_{AII} | T7/M3_R + M1_F/T7T | N_{H23}MC_{AII} + | pEM3BT2 |
| | | N_{YIII}M₃C_{AII} | |
| N_{YIII}M₄C_{AII} | T7/M3_R + M1_F/T7T | N_{YIII}MC_{AII} + | pEM3BT2 |
| | | N_{YIII}M₃CA_{II} | |
| N_{A4}M₄C_{AII} | T7/M3_R + M1_F/T7T | N_{A4}MC_{AII} + | pEM3BT2 |
| | | N_{YIII}M₃C_{AII} | |
| N_{A5}M₄C_{AII} | T7/M3_R + M1_F/T7T | N_{A5}MC_{AII} + | pEM3BT2 |
| | | N_{YIII}M₃C_{AII} | |
| N_{A6}M4C_{AII} | T7/M3_R + M1_F/T7T | N_{A6}MC_{AII} + | pEM3BT2 |
| | | N_{YIII}M₃C_{AII} | |
| N_{A7}M₄C_{AII} | T7/M3_R + M1_F/T7T | N_{A7}MC_{AII} + | pEM3BT2 |
| | | N_{YIII}M₃C_{AII} | |
| N_{A8}M₄C_{AII} | T7/M3_R + M1_F/T7T | N_{A8}MC_{AII} + | pEM3BT2 |
| | | N_{YIII}M₃C_{AII} | |
| N_{A9}M₄C_{AII} | T7/M3_R + M1_F/T7T | N_{A9}MC_{AII} + | pEM3BT2 |
| | | N_{YIII}M₃C_{AII} | |
| MNG-3BTC | mNG-3BTC F/mNG- | mNeonGreen | pEM3BTC |
| | 3BTC_R | | |
| (KR)₅- | KR5_Top/KR5_Bot | — | mNeonGreen- |
| mNeonGreen | | | 3BTC |

TABLE 3

| | | SEQ ID |
|---|---|---|
| Name | Sequence | NO: |
| H23MC_Fwd | 5′ -AAAGCTCTTCACAGGGCGCCCTTCCAGCCC | 33 |
| H23MC_Rev | 5′ -GCTTTGTTAGCAGCCGGATC | 34 |
| 3BTC_Fwd | 5′ - | 35 |
| | CGAAAGCAGCGGCCTGGAAGTGCTGTTTCAGGGTCCGAGAAGAGCCATGGC | |
| 3BTC_Rev | 5′ - | 36 |
| | GCCATGGCTCTTCTCGGACCCTGAAACAGCACTTCCAGGCCGCTGCTTTCG | |
| mNG-3BTC_F | 5′ - | 37 |
| | AAAGCTCTTCACCGGGATCCAAAAGTGGTCTCGGCGCCGGCTCGAAGGGGG | |
| | AAGAAGATAAC | |
| mNG-3BTC_R | 5′ -AAAAGATCTTTATTACTTATAAAGCTCATCCATGCCC | 38 |
| Y_Rev | 5′ -AAAGCTCTTCAACCGCTTGCAATCTGTGAGAG | 39 |
| M3_Fwd | 5′ -AAAGCTCTTCAGGCGGTAACGAGCAGATTCAGGC | 40 |
| V1_Fwd | 5′- | 41 |
| | AAAGCTCTTCAGTGAAGTTACTGAAAAGCTCTAACGAACAGATTCTCCAAG | |
| | AGG | |
| V1_Rev | 5′ | 42 |
| | AAAGCTCTTCACACCAGTTTAGGCAGATCCGGACCCTGGAAGTACAGGTTT | |
| | TCGC | |
| V41 Fwd | 5′ - | 43 |
| | AAAGCTCTTCACTGCGTGCACTCGCTGAAATTGCCAGCGGCGGTAACGAGC | |
| | AGATTC | |
| V41_Rev | 5′ - | 44 |
| | AAAGCTCTTCACAGCGCTTTCAGCAGGATTTCCTCGTTAGAGCTTTTCAGT | |
| | AACTTCACC | |
| V42_Fwd | 5′ - | 45 |
| | AAAGCTCTTCACTGAAGGCACTCGCTGAAATTGCCAGCGGCGGTAACGAGC | |
| | AGATTC | |
| V5_Fwd | 5′ - | 46 |
| | AAAGCTCTTCACTGAAGACACTCGCTGAAATTGCCAGCGGCGGTAACGAGC | |
| | AGATTC | |
| V5_Rev | 5′ - | 47 |
| | AAAGCTCTTCACAGCGCTTTCAGCAGGGTTTCCTCATCAGGTGACGAAAGC | |
| | AATTGGAC | |
| V6_Fwd | 5′ - | 48 |
| | AAAGCTCTTCACTGCGTACACTCGCTGAAATTGCCAGCGGCGGTAACGAGC | |
| | AGATTC | |
| V6_Rev | 5′ - | 49 |
| | AAAGCTCTTCACAGCGCTTTCAGCAGGGTTTCCTCATCAGAGCTTTTCAGT | |
| | AACTTCACC | |
| V8_Rev | 5′ - | 50 |
| | AAAGCTCTTCACAGCGCTTCCAGCAGGGTTTTCTCATCAGAGCTTTTCAGT | |
| | AACTTCACC | |
| M3_R | 5′ -AAAGCTCTTCACCCACCAGAGGCAATGTTAG | 51 |
| M1_F | 5′ -AAAGCTCTTCAGGGAATGAGCAAATCCAAGCCGTG | 52 |
| T7 | 5′ -TAATACGACTCACTATAGGG | 53 |
| T7T | 5′ -GCTAGTTATTGCTCAGCGG | 54 |

TABLE 4

| | |
|---|---|
| Wavelength | 1.000 |
| Resolution range (Å) | 41.06-1.59 (1.65-1.59) |
| Space group | P212121 |
| Unit cell | |
| a, b, c (Å) | 56.59, 62.66, 108.74 |
| α, β, γ (°) | 90, 90, 90 |
| Total reflections | 706321 (71586) |
| Unique reflections | 52752 (5191) |
| Multiplicity | 13.4 (13.8) |
| Completeness (%) | 99.96 (99.98) |
| Mean I/sigma(I) | 22.36 (1.48) |
| Wilson B-factor | 28.93 |
| R-merge | 0.056 (1.382) |
| R-meas | 0.059 (1.435) |
| R-pim | 0.016 (0.384) |
| CC1/2 | 1 (0.702) |
| CC* | 1 (0.908) |
| ISa | 30.57 |
| Reflections used in refinement | 52751 (5190) |
| Reflections used for R-free | 2637 (259) |
| R-work | 0.186 (0.437) |
| R-free | 0.214 (0.423) |
| CC(work) | 0.964 (0.811) |
| CC(free) | 0.948 (0.761) |
| Number of non-hydrogen atoms | 3282 |
| Macromolecules | 2922 |
| Ligands | 34 |
| Solvent | 326 |
| Protein residues | 369 |
| RMS(bonds) | 0.029 |
| RMS(angles) | 1.92 |
| Ramachandran favored (%) | 99.45 |
| Ramachandran allowed (%) | 0.55 |
| Ramachandran outliers (%) | 0.00 |
| Rotamer outlier (%) | 0.32 |
| Clashscore | 6.24 |
| Average B-factor | 36.42 |
| Macromolecules | 35.01 |
| Ligands | 52.50 |
| Solvent | 47.43 |
| Number of TLS groups | 2 |

Statistics for the highest-resolution shell are shown in parentheses.

TABLE 5

| N_{H23}MC_{AII} | Rosetta | ΔREU |
|---|---|---|
| Residue | Suggestion | (Rosetta-Original) |
| Gly | Pro | −0.663 |
| Ala | Asp | 1.179 |
| Leu | Leu | — |
| Pro | Pro | — |
| Ala | Lys | −0.51 |
| Leu | Leu | — |
| Val | Val | — |
| Gln | Lys | −0.438 |
| Leu | Leu | — |
| Leu | Leu | — |
| Ser | Lys | −0.707 |
| Ser | Ser | — |
| Pro | Asn | 0.15 |
| Asn | Asp | 0.987 |
| Glu | Glu | — |
| Gln | Lys | −0.229 |
| Ile | Glu | −0.289 |
| Leu | Leu | — |
| Gln | Leu | −1.801 |
| Glu | Glu | — |
| Ala | Ala | — |
| Leu | Leu | — |
| Trp | Arg | −3.487 |
| Ala | Thr | 0.178 |
| Leu | Leu | — |
| Ser | Ala | −1.926 |
| Asn | Val | −0.805 |
| Ile | Ile | — |
| Ala | Ala | 0.004 |
| Ser | Ser | −0.007 |

TABLE 6

| | N_{YIII}-Cap | | N_{A4}-Cap | | ΔREU |
|---|---|---|---|---|---|
| Position | Residue | [REU] | Residue | [REU] | N_{A4}-N_{YIII} |
| 1 | Gly | −0.12 | Pro | 1.74 | 1.86 |
| 2 | Glu | −1.13 | Asp | −0.35 | 0.78 |
| 3 | Leu | −1.85 | Leu | −3.26 | −1.41 |
| 4 | Pro | −0.60 | Pro | 0.28 | 0.88 |
| 5 | Gln | 1.78 | Lys | 0.93 | −0.85 |
| 6 | Met | −1.72 | Leu | −5.14 | −3.42 |
| 7 | Val | −5.72 | Val | −6.02 | −0.30 |
| 8 | Gln | 1.60 | Lys | 1.27 | −0.33 |
| 9 | Gln | −1.14 | Leu | −3.90 | −2.76 |
| 10 | Leu | −5.52 | Leu | −5.22 | 0.30 |
| 11 | Asn | 0.10 | Lys | 0.96 | 0.86 |
| 12 | Ser | −1.08 | Ser | −0.47 | 0.61 |
| 13 | Pro | 0.90 | Ser | −0.85 | −1.75 |
| 14 | Asp | −1.12 | Asn | −1.80 | −0.68 |
| 15 | Gln | 0.41 | Glu | 0.20 | −0.21 |
| 16 | Gln | 1.29 | Glu | 1.53 | 0.24 |
| 17 | Glu | −1.08 | Ile | −2.90 | −1.82 |
| 18 | Leu | −4.69 | Leu | −4.48 | 0.21 |
| 19 | Gln | 0.21 | Leu | −3.32 | −3.53 |
| 20 | Ser | 0.55 | Lys | −0.10 | −0.65 |
| 21 | Ala | −4.35 | Ala | −4.98 | −0.63 |
| 22 | Leu | −6.30 | Leu | −6.70 | −0.40 |
| 23 | Arg | 0.81 | Arg | 0.18 | −0.63 |
| 24 | Lys | −0.09 | Ala | −3.81 | −3.72 |
| 25 | Leu | −6.67 | Leu | −6.84 | −0.17 |
| 26 | Ser | −1.73 | Ala | −6.97 | −5.24 |
| 27 | Gln | 0.79 | Glu | 1.17 | 0.38 |
| 28 | Ile | −2.00 | Ile | −3.20 | −1.20 |
| 29 | Ala | −4.04 | Ala | −4.07 | −0.03 |
| 30 | Ser | 0.45 | Ser | 0.64 | 0.19 |

TABLE 7

| NM₄C variant | K_{d} ± St. Dev. [nM] |
|---|---|
| N_{YIII}-M₄C | 36.1 ± 2.9 |
| N_{A4}-M₄C | 30.5 ± 2.3 |
| N_{A5}-M₄C | 48.6 ± 10.7 |
| N_{A6}-M₄C | 29.9 ± 5.6 |
| N_{A7}-M₄C | 28.7 ± 6.4 |
| N_{A8}-M₄C | 22.9 ± 5.1 |
| N_{A9}-M₄C | 45.1 ± 3 |

1. An armadillo repeat protein comprising or essentially consisting of
a. an N-terminal cap sequence;
b. a C-terminal cap sequence; and
c. a plurality of armadillo repeats,
wherein each armadillo repeat comprises three helices a, b, and c, wherein the helices a and b are connected via a loop a/b, and the helices b and c are connected via a loop b/c, and wherein two armadillo repeats are connected via a loop c/a;
wherein
the C-terminal cap sequence consists of a sequence NEQIQAVIDAGALEKLEQLQSHENEKIQKEAQEALEKLQSH (SEQ ID NO: 2);
helix a consists of a sequence X⁷EQIQAVIDA (SEQ ID NO: 3);
loop a/b consists of a single glycine G;
helix b consists of a sequence ALPALVQLLS (SEQ ID NO: 4),
loop b/c consists of a sequence serine proline SP;
helix c consists of a sequence NEX¹ILX²X³ALX⁴ALX⁵NIAX⁶ (SEQ ID NO: 5); and
loop c/a consist of 1 to 9 proteinogenic amino acids;
wherein each X¹-X⁷ can be any proteinogenic amino acid provided that the amino acid does not prevent helix formation of helix a and c;
the armadillo repeat protein being characterized in that
the N-terminal cap sequence consists of the sequence X₀X₁LX₃X₄LVX₇LLX₁₀X₁₁X₁₂X₁₃X₁₄X₁₅X₁₆LLX₁₉ALX₂₂X₂₃L AX₂₆IAX₂₉ (SEQ ID NO: 1);
wherein the variables of SEQ ID NO: 1 can take the following values:
X₀: any proteinogenic amino acid sequence of 1-10 amino acids, wherein the sequence is capable of forming a helix;
X₁: any proteinogenic amino acid, particularly an amino acid selected from D, E, and A;
X₃: any proteinogenic amino acid, particularly P;
X₄: an amino acid selected from K, R, Q, N, D, E, A, L, and M;
X₇: an amino acid selected from K, R, Q, N, D, E, A, L, and M;
X₁₀: an amino acid selected from K, R, Q, N, D, E, A, L, and M;
X₁₁₋₁₃: independently any proteinogenic amino acid, wherein 1, 2, 3, 4, or 5 amino acids may be inserted additionally into X₁₁₋₁₃, particularly X₁₁₋₁₃ are independently selected from S, T, G, P, N, and D;
X₁₄: an amino acid selected from K, R, Q, N, D, E, A, L, and M;
X₁₅: an amino acid selected from K, R, Q, N, D, E, A, L, and M;
X₁₆: an amino acid selected from I, E, and T;
X₁₉: an amino acid selected from K, R, Q, N, D, E, A, L, and M;
X₂₂: an amino acid selected from K, R, Q, N, D, E, A, L, and M;
X₂₃: an amino acid selected from A, K, T, R, Q, N, D, E, A, L, and M;
X₂₆: an amino acid selected from K, R, Q, N, D, E, A, L, and M;
X₂₉: 1-20, particularly 1-10, amino acids independently selected from any proteinogenic amino acid provided that the amino acid does not prevent loop formation;
wherein optionally, the C-terminal cap sequence and the plurality of armadillo repeats may be varied:
a total of 1, 2, or 3 amino acids per armadillo repeat may be inserted at the beginning or the end of the helices forming one repeat, or inside the loops, and/or
1, 2, or 3 amino acids per armadillo repeat and per C-terminal cap sequence may be exchanged, particularly according to the following substitution rules:
a. glycine (G), serine(S), and alanine (A) are interchangeable; valine (V), leucine (L), and isoleucine (I) are interchangeable, A and V are interchangeable;
b. tryptophan (W) and phenylalanine (F) are interchangeable, tyrosine (Y) and F are interchangeable;
c. serine(S) and threonine (T) are interchangeable;
d. aspartic acid (D) and glutamic acid (E) are interchangeable
e. asparagine (N) and glutamine (Q) are interchangeable; N and S are interchangeable; N and D are interchangeable; E and Q are interchangeable;
f. methionine (M) and Q are interchangeable;
g. cysteine (C), A, V and S are interchangeable;
h. proline (P), G, S and A are interchangeable;
i. arginine (R) and lysine (K) are interchangeable;
j. salt bridge partners are interchangeable.

2. The armadillo repeat protein according to claim 1, wherein the N-terminal cap consists of the sequence X₀X₁LX₃X₄LVX₇LLX₁₀X₁₁X₁₂X₁₃X₁₄X₁₅X₁₆LLX₁₉ALX₂₂X₂₃LAX₂₆IAX₂₉ (SEQ ID NO: 1), wherein
X₀: any proteinogenic amino acid sequence of 1-10 amino acids, wherein the sequence is capable of forming a helix;
X₁: any proteinogenic amino acid, particularly an amino acid selected from D and A;
X₃: any proteinogenic amino acid, particularly P;
X₄: an amino acid selected from K, Q, A, and E;
X₇: an amino acid selected from K and E;
X₁₀: an amino acid selected from K, S, N, A, and E;
X₁₁₋₁₃: independently any proteinogenic amino acid, wherein 1, 2, 3, 4, or 5 amino acids may be inserted additionally in X₁₁₋₁₃, particularly
X₁₁ is selected from S, G, D and N,
X₁₂ is selected from S, T, G, P, N and D, and
X₁₃ is selected from N and D;
X₁₄: an amino acid selected from K, R, Q, E, A, and L;
X₁₅: an amino acid selected from K, R, Q, E, A, and L;
X₁₆: an amino acid selected from I, E, and T;
X₁₉: an amino acid selected from K, R, Q, E, A, and L;
X₂₂: an amino acid selected from K, R, Q, E, A, and L;
X₂₃: an amino acid selected from K, R, Q, E, A, L and T;
X₂₆: an amino acid selected from K, R, Q, E, A, and L;
X₂₉: 1-20, particularly 1-10, amino acids selected from any proteinogenic amino acid provided that the amino acid does not prevent loop formation.

3. The armadillo repeat protein according to claim 1, wherein the N-terminal cap consists of the sequence X₀X₁LX₃X₄LVX₇LLX₁₀SX₁₂X₁₃EX₁₅X₁₆LLX₁₉ALX₂₂X₂₃LAX₂₆IAX₂₉ (SEQ ID NO: 56), wherein
X₀: any proteinogenic amino acid sequence of 1-10 amino acids, wherein the sequence is capable of forming a helix;
X₁: any proteinogenic amino acid selected from D and A;
X₃: any proteinogenic amino acid, particularly P;
X₄: an amino acid selected from K, A, and E;
X₇: an amino acid selected from K and E;
X₁₀: an amino acid selected from K, E, and S;
X₁₂: any proteinogenic amino acid provided that the amino acid does not prevent loop formation, particularly S;
X₁₃: an amino acid selected from N and D;
X₁₅: an amino acid selected from E and K;
X₁₆: an amino acid selected from I and T;
X₁₉: an amino acid selected from K and E;
X₂₂: an amino acid selected from K and R;
X₂₃: an amino acid selected from A and T;
X₂₆: an amino acid selected from E and Q;
X₂₉: 1-20, particularly 1-10, amino acids independently selected from any proteinogenic amino acid provided that the amino acid does not prevent loop formation.

4. The armadillo repeat protein according to claim 1, wherein the N-terminal cap sequence is selected from a sequence in the following table

| N-Cap | | |
|---|---|---|
| Variant | Sequence | SEQ ID NO: |
| NA4 | PDLPKLVKLLKSSNEEILLKALRALAEIASGG | 6 |
| NA5 | PDLPKLVKLLKSSNEEILLKALKALAEIASGG | 7 |
| NA6 | GALPALVQLLSSPDEETLLKALKTLAEIASGG | 8 |
| NA7 | PDLPKLVKLLKSSDEETLLKALRTLAEIASGG | 9 |
| NA8 | PDLPKLVKLLKSSDEETLLKALKTLAEIASGG | 10 |
| NA9 | PDLPKLVKLLKSSDEKTLLEALKTLAEIASGG | 11 |

wherein optionally, the N-terminal cap sequence may be varied:
a total of 1, 2, or 3 amino acids per N-terminal cap sequence may be inserted, and/or
a total of 1, 2, or 3 amino acids per N-terminal cap sequence may be removed, and/or
1, 2, or 3 amino acids per N-terminal cap sequence may be exchanged, particularly according to the substitution rules listed in claim 1.

5. The armadillo repeat protein according to claim 1, wherein the N-terminal cap sequence is selected from a sequence of the group consisting of SEQ ID NO 6 to SED ID NO 10, wherein optionally, 1, 2, or 3 amino acids per N-terminal cap sequence may be exchanged, particularly according to the substitution rules listed in claim 1.

6. The armadillo repeat protein according to claim 1, wherein the N-terminal cap sequence is selected from a sequence of the group consisting of SEQ ID NO 6 to SED ID NO 10.
